(19) United States
(12) Patent Application Publication
(10) Pub. No.: US 2026/0217644 A1
(43) Pub. Date: Jul. 30, 2026

(54) CRYSTAL FORMS OF AMINO LIPIDS

(71) Applicant: ModernaTX, Inc., Cambridge, MA (US)

(72) Inventors: Orn ALMARSSON, Cambridge, MA (US); Eugene Cheung, Cambridge, MA (US)

(21) Appl. No.: 19/446,578

(22) Filed: Jan. 12, 2026

Related U.S. Application Data
(63) Continuation of application No. 17/525,352, filed on Nov. 12, 2021, now Pat. No. 12,552,738.

(51) Int. Cl. C07C 229/12; C07C 55/07; C07C 65/03; C07C 227/16; C07C 229/24

(52) U.S. Cl. CPC C07C 229/12; C07C 55/07; C07C 65/03; C07C 227/16; C07C 229/24; C07B 2200/13

(57) ABSTRACT

Provided herein are novel solid forms of each of four compounds: (1) heptadecan-9-yl 8-((2-hydroxyethyl)amino)octanoate (“Compound 1”), (2) heptadecan-9-yl 8-((2-hydroxyethyl)(6-oxo-6-(undecyloxy)hexyl)amino)octanoate (“Compound 2”), (3) heptadecan-9-yl 8-((2-hydroxyethyl)(8-(nonyloxy)-8-oxooctyl)amino)octanoate (“Compound 3”), and (6Z,9Z,28Z,31Z)-heptatriaconta-6,9,28,31-tetraen-19-yl 4-(dimethylamino)butanoate (“MC3”), and related compositions and methods.

### RELATED APPLICATIONS

This application is a division of U.S. application Ser. No. 16/493,789, filed Sep. 13, 2019, which is a U.S. National Phase application, filed under U.S.C. § 371, of International Application No. PCT/US2018/022740, filed Mar. 15, 2018, which claims priority to, and the benefit of, U.S. Provisional Application No. 62/471,908, filed Mar. 15, 2017; the entire content of which is incorporated herein by reference.

### TECHNICAL FIELD

This disclosure relates to solid crystalline forms of each of three compounds: (1) heptadecan-9-yl 8-((2-hydroxyethyl)amino)octanoate (“Compound 1”), (2) heptadecan-9-yl 8-((2-hydroxyethyl)(6-oxo-6-(undecyloxy)hexyl)amino)octanoate (“Compound 2”), and (3) heptadecan-9-yl 8-((2-hydroxyethyl)(8-(nonyloxy)-8-oxooctyl)amino)octanoate (“Compound 3”), and related compositions and methods. This disclosure also relates to solid crystalline forms of (6Z,9Z,28Z,31Z)-heptatriaconta-6,9,28,31-tetraen-19-yl 4-(dimethylamino)butanoate (“MC3”), and related compositions and methods.

### BACKGROUND

The effective targeted delivery of biologically active substances such as small molecule drugs, proteins, and nucleic acids represents a continuing medical challenge. In particular, the delivery of nucleic acids to cells is made difficult by the relative instability and low cell permeability of such species. Thus, there exists a need to develop methods and compositions to facilitate the delivery of therapeutic and/or prophylactics such as nucleic acids to cells.

Lipid-containing nanoparticle compositions, liposomes, and lipoplexes have proven effective as transport vehicles into cells and/or intracellular compartments for biologically active substances such as small molecule drugs, proteins, and nucleic acids. Such compositions generally include one or more “cationic” and/or amino (ionizable) lipids, phospholipids including polyunsaturated lipids, structural lipids (e.g., sterols), and/or lipids containing polyethylene glycol (PEG lipids). Cationic and/or ionizable lipids include, for example, amine-containing lipids that can be readily protonated. Though a variety of such lipid-containing nanoparticle compositions have been demonstrated, improvements in safety, efficacy, and specificity are still lacking. In addition, the physical and chemical properties of lipid materials often present challenges relating to the practice of making and using lipid-containing nanoparticles for drug delivery.

### SUMMARY

Long-chain amino lipids are usually viscous oils at room temperature. Solid forms of these lipids are desirable for e.g., improving handling, improving stability (such as storage stability) and/or control of physical/chemical properties, simplifying purification process, simplifying large-scale production process and/or increasing accuracy in measurements and characterization of lipids.

Accordingly, provided herein are novel solid forms (e.g., crystalline forms) of each of three compounds (1) heptadecan-9-yl 8-((2-hydroxyethyl)amino)octanoate (“Compound 1”), (2) heptadecan-9-yl 8-((2-hydroxyethyl)(6-oxo-6-(undecyloxy)hexyl)amino)octanoate (“Compound 2”), and (3) heptadecan-9-yl 8-((2-hydroxyethyl)(8-(nonyloxy)-8-oxooctyl)amino)octanoate (“Compound 3”), the structure of each of which is provided below:

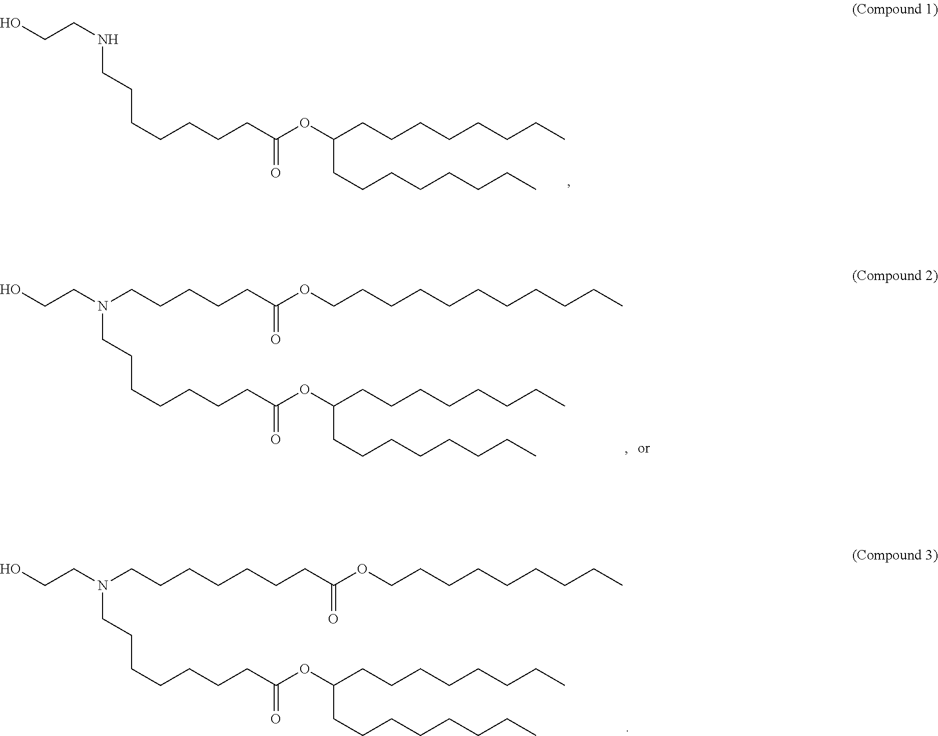 [SMILES: CCCCCCCCC(CCCCCCCC)OC(=O)CCCCCCCNCCO ; CCCCCCCCCCCOC(=O)CCCCCN(CCO)CCCCCCCC(=O)OC(CCCCCCCC)CCCCCCCC ; CCCCCCCCCOC(=O)CCCCCCCN(CCO)CCCCCCCC(=O)OC(CCCCCCCC)CCCCCCCC]

In another aspect, provided herein are novel solid forms (e.g., crystalline forms) of (6Z,9Z,28Z,31Z)-heptatriaconta-6,9,28,31-tetraen-19-yl 4-(dimethylamino)butanoate (“MC3”), the structure of which is provided below:

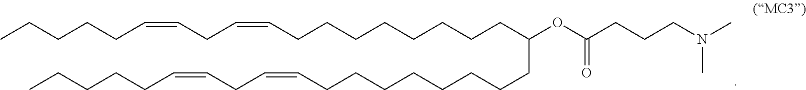 [SMILES: CCCCC/C=C\C/C=C\CCCCCCCCC(CCCCCCCC/C=C\C/C=C\CCCCC)OC(=O)CCCN(C)C]

In one aspect, disclosed herein is salt or cocrystal of heptadecan-9-yl 8-((2-hydroxyethyl)amino)octanoate (“Compound 1”), heptadecan-9-yl 8-((2-hydroxyethyl)(6-oxo-6-(undecyloxy)hexyl)amino)octanoate (“Compound 2”), or heptadecan-9-yl 8-((2-hydroxyethyl)(8-(nonyloxy)-8-oxooctyl)amino)octanoate (“Compound 3”). In another aspect, the salt or cocrystal of Compound 1, 2, or 3 has a melting point of about 50° C. or greater (e.g., about 60° C., about 70° C. or greater). In another aspect, the salt or cocrystal of Compound 3 has a melting point of about 270° C. or greater (e.g., about 280° C., about 290° C. or greater). For example, the salt or cocrystal of Compound 1, 2, or 3 is formed between Compound 1, 2, or 3 and a coformer compound (e.g., an acid).

In one aspect, disclosed herein is a salt or cocrystal of (6Z,9Z,28Z,31Z)-heptatriaconta-6,9,28,31-tetraen-19-yl 4-(dimethylamino)butanoate (“MC3”). In another aspect, the salt or cocrystal of MC3 has a melting point of about 150° C. or greater (e.g., about 160° C., about 170° C., about 180° C. or greater, about 190° C. or greater). In another aspect, disclosed herein is a salt or cocrystal of (6Z,9Z,28Z,31Z)-heptatriaconta-6,9,28,31-tetraen-19-yl 4-(dimethylamino)butanoate (“MC3”). In another aspect, the salt or cocrystal of MC3 has a melting point of about 50° C. or greater (e.g., about 60° C., about 70° C., about 80° C. or greater). For example, the salt or cocrystal of MC3 is formed between MC3 and a coformer compound (e.g., an acid).

In one aspect, this disclosure is directed to a salt or cocrystal of heptadecan-9-yl 8-((2-hydroxyethyl)amino)octanoate (“Compound 1”) and a compound (e.g., a coformer compound) selected from the group consisting of 4-hydroxybenzoic acid, oxalic acid, trimellitic acid, orotic acid, trimesic acid, and sulfuric acid.

In another aspect, this disclosure is directed to a salt or cocrystal of heptadecan-9-yl 8-((2-hydroxyethyl)(6-oxo-6-(undecyloxy)hexyl)amino)octanoate (“Compound 2”) and a compound (e.g., a coformer compound) selected from the group consisting of trimesic acid, (−)-2,3-dibenzoyl-L-tartaric acid, 4-acetamido benzoic acid, (+)-L-tartaric acid, and methanesulfonic acid.

In yet another aspect, this disclosure is directed to a salt or cocrystal of heptadecan-9-yl 8-((2-hydroxyethyl)(8-(nonyloxy)-8-oxooctyl)amino)octanoate (“Compound 3”) and trimesic acid.

In one aspect, this disclosure is directed to a salt or cocrystal of (6Z,9Z,28Z,31Z)-heptatriaconta-6,9,28,31-tetraen-19-yl 4-(dimethylamino)butanoate (“MC3”) and a compound selected from the group consisting of (+)-O,O-di-pivaloyl-D-tartaric acid (DPDT), (−)-O,O-di-pivaloyl-L-tartaric acid (DPLT), (+)-2,3-dibenzoyl-D-tartaric acid (DBDT), and trimesic acid. In one embodiment this disclosure is directed to a salt or cocrystal of (6Z,9Z,28Z,31Z)-heptatriaconta-6,9,28,31-tetraen-19-yl 4-(dimethylamino)butanoate (“MC3”) and trimesic acid.

The salts or cocrystals disclosed herein may comprise Compound 1 (or Compound 2 or 3) and the coformer compound (e.g., an acid), within a ratio of from about 1:0.2 mol/mol (i.e., 5:1 mol/mol) to 1:5 mol/mol or from about 1:0.5 mol/mol (i.e., 2:1 mol/mol) to 1:2 mol/mol, or within the range of from 1:0.4 mol/mol (i.e., 2.5:1 mol/mol) to 1:1.1 mol/mol.

The salts or cocrystals disclosed herein may comprise (6Z,9Z,28Z,31Z)-heptatriaconta-6,9,28,31-tetraen-19-yl 4-(dimethylamino)butanoate (“MC3”) and the coformer compound (e.g., an acid), within a ratio of from about 1:0.5 mol/mol (i.e., 2:1 mol/mol) to 1:2 mol/mol. For example the ratio is about 1:1.2 mol/mol, about 1:1.1 mol/mol, or about 1:1.5 mol/mol).

The salts or cocrystals disclosed herein may be anhydrous and/or essentially solvent-free form, or be in hydrate and/or solvate form. For example, 4-hydroxybenzoate of Compound 1 is anhydrous. For example, Compound 1 orotate may be anhydrous or in a hydrate or solvate form. For example, trimesate of MC3 may be anhydrous or in a hydrate or solvate form.

The salts or cocrystals disclosed herein may be non-hygroscopic. For example, the 4-hydroxybenzoate of Compound 1 is non-hygroscopic. For example, the trimesate of MC3 is non-hygroscopic.

It has been found that under suitable conditions some of the salts or cocrystals can be obtained in the form of different polymorphs. For example, 4-hydroxybenzoate of Compound 1 has at least two polymorphs, Polymorphs A and B. For example, orotate of Compound 1 has at least two polymorphs, Polymorphs A and B. For example, orotate of Compound 7 has at least two polymorphs, Polymorphs A and B. For example trimesate of Compound 3 has at least two polymorphs, Polymorphs A and B. For example, trimesate of MC3 has at least two polymorphs, Polymorphs A and B

The polymorphs disclosed herein may be substantially pure, i.e., substantially free of impurities. Non-limiting examples of impurities include other polymorph forms, or residual organic and inorganic molecules such as related impurities (e.g., intermediates used to make the compounds), solvents, water or salts. As used herein “substantially pure” or “substantially free of impurities” means there is not a significant amount of impurities (e.g., other polymorph forms, or residual organic and inorganic molecules such as related impurities, solvents, water or salts) present in a sample of the salt, cocrystal, or polymorph. For example, a salt, cocrystal, or polymorph disclosed herein contains less than 10% weight by weight (wt/wt) total impurities, less than 5% wt/wt total impurities, less than 2% wt/wt total impurities, less than 1% wt/wt total impurities, less than 0.5% wt/wt total impurities, or not a detectable amount of impurities.

In one embodiment, Polymorph A of 4-hydroxybenzoate of Compound 1 is substantially free of impurities, meaning there is not a significant amount of impurities present in the sample of Polymorph A. In another embodiment, Polymorph A is a crystalline solid substantially free of Compound 1 (or any of its amorphous salt forms). In yet another embodiment, Polymorph A is a crystalline solid substantially free of other polymorphs of 4-hydroxybenzoate of Compound 1 and substantially free of amorphous Compound 1 (or any of its amorphous salt forms). For example, Polymorph A is a crystalline solid substantially free of Polymorph B of 4-hydroxybenzoate of Compound 1 and substantially free of amorphous Compound 1 (or any of its amorphous salt forms). The skilled artisan understands that a solid sample of Polymorph A may also include other polymorphs (e.g., Polymorph B), and/or amorphous Compound 1 (or any of its amorphous salt forms).

Polymorph A of 4-hydroxybenzoate of Compound 1 can be defined according to its X-ray powder diffraction pattern. Accordingly, in one embodiment, Polymorph A exhibits an X-ray powder diffraction pattern obtained using Cu Kα radiation, having two, three, or more characteristic peaks expressed in degrees 2-theta (+/−0.2) selected from the group consisting of 4.5, 6.8, 9.1, and 11.4. In one embodiment, Polymorph A exhibits an X-ray powder diffraction pattern obtained using Cu Kα radiation, having peaks with 2-theta values substantially in accordance with FIG. 1. In another embodiment, Polymorph A exhibits an X-ray powder diffraction pattern obtained using Cu Kα radiation, having peaks with 2-theta values substantially in accordance with Table I.

Polymorph A of 4-hydroxybenzoate of Compound 1 can also be defined according to its differential scanning calorimetry thermogram. In one embodiment, the polymorph exhibits a differential scanning calorimetry thermogram showing a primary endotherm expressed in units of ° C. at a temperature of 103+/−2° C. and a second primary endotherm expressed in units of ° C. at a temperature of 68+/−2° C. In another embodiment, Polymorph A exhibits a differential scanning calorimetry thermogram substantially in accordance with the lower curve shown in FIG. 3.

In one embodiment, Polymorph B of Compound 1 orotate is substantially free of impurities (e.g., phase or form impurities), meaning there is not a significant amount of impurities present in the sample of Polymorph B. In another embodiment, Polymorph B is a crystalline solid substantially free of amorphous Compound 1 (or any of its amorphous salt forms). In yet another embodiment, Polymorph B is a crystalline solid substantially free of other polymorphs of Compound 1 orotate and substantially free of amorphous Compound 1 (or any of its amorphous salt forms). For example, Polymorph B is a crystalline solid substantially free of Polymorph A of Compound 1 orotate and substantially free of amorphous Compound 1 (or any of its amorphous salt forms). The skilled artisan understands that a solid sample of Polymorph B of Compound 1 orotate may also include other polymorphs (e.g., Polymorph A), and/or amorphous Compound 1 (or any of its amorphous salt forms).

Polymorph B of Compound 1 orotate can be defined according to its X-ray powder diffraction pattern. Accordingly, in one embodiment, Polymorph B exhibits an X-ray powder diffraction pattern obtained using Cu Kα radiation, having two, three, four, or more characteristic peaks expressed in degrees 2-theta (+/−0.2) selected from the group consisting of 5.1, 7.5, 10.1, 12.7, 15.2, and 17.8. In one embodiment, Polymorph B exhibits an X-ray powder diffraction pattern obtained using Cu Kα radiation, having peaks with 2-theta values substantially in accordance with FIG. 18, upper profile. In another embodiment, Polymorph B exhibits an X-ray powder diffraction pattern obtained using Cu Kα radiation, having peaks with 2-theta values substantially in accordance with Table III.

In one embodiment, Polymorph B of trimesate of Compound 3 is substantially free of impurities, meaning there is not a significant amount of impurities present in the sample of Polymorph B. In another embodiment, Polymorph B is a crystalline solid substantially free of Compound 3 (or any of its amorphous salt forms). In yet another embodiment, Polymorph B is a crystalline solid substantially free of other polymorphs of trimesate of Compound 3 and substantially free of amorphous trimesate of Compound 3 (or any of its amorphous salt forms). For example Polymorph B is a crystalline solid substantially free of Polymorph A of trimesate of Compound 3 and substantially free of amorphous trimesate of Compound 3 (or any of its amorphous salt forms). The skilled artisan understands that a solid sample of Polymorph B may also include other polymorphs (e.g., Polymorph A) and/or amorphous Compound 3 (or any of its amorphous salt forms).

Polymorph B of Compound 3 trimesate can be defined according to its X-ray powder diffraction pattern. Accordingly, Polymorph B of Compound 3 trimesate exhibits an X-ray powder diffraction pattern obtained using Cu Kα radiation, having two, three, four or more characteristic peaks expressed in degrees 2-theta (+/−0.4) at 6.2, 10.8, 16.5, and 26.7. In one embodiment, Polymorph B exhibits an X-ray powder diffraction pattern obtained using Cu Kα radiation, having peaks with 2-theta values substantially in accordance with FIG. 48. In another embodiment, Polymorph B exhibits an X-ray powder diffraction pattern obtained using Cu Kα radiation, having peaks with 2-theta values substantially in accordance with Table XII.

In other embodiments, Polymorph B of trimesate of Compound 3 is identifiable on the basis of a characteristic peak observed in a differential scanning calorimetry thermogram. In one embodiment, the polymorph exhibits a differential scanning calorimetry thermogram showing a characteristic melting endotherm peak expressed in units of ° C. with an onset temperature of about 305+/−2° C. In another embodiment, the polymorph exhibits a differential scanning calorimetry thermogram showing a second primary endotherm expressed in units of ° C. at a temperature of 240+/−2° C. In another embodiment, the polymorph exhibits a differential scanning calorimetry thermogram substantially in accordance with FIG. 49.

In one embodiment, Polymorph A of trimesate of MC3 is substantially free of impurities, meaning there is not a significant amount of impurities present in the sample of Polymorph A. In another embodiment, Polymorph A is a crystalline solid substantially free of MC3 (or any of its amorphous salt forms). In yet another embodiment, Polymorph A is a crystalline solid substantially free of other polymorphs of trimesate of MC3 and substantially free of amorphous MC3 (or any of its amorphous salt forms). For example, Polymorph A is a crystalline solid substantially free of Polymorph B of trimesate of MC3 and substantially free of amorphous MC3 (or any of its amorphous salt forms). The skilled artisan understands that a solid sample of Polymorph A may also include other polymorphs (e.g., Polymorph B), and/or amorphous MC3 (or any of its amorphous salt forms).

Polymorph A of MC3 trimesate can be defined according to its X-ray powder diffraction pattern. Accordingly, Polymorph A of MC3 trimesate exhibits an X-ray powder diffraction pattern obtained using Cu Kα radiation, having two, three, four or more characteristic peaks expressed in degrees 2-theta (+/−0.4) at 5.2, 7.8, 10.4, 18.3, 20.9, 23.6, or 26.2. In one embodiment, Polymorph A exhibits an X-ray powder diffraction pattern obtained using Cu Kα radiation, having peaks with 2-theta values substantially in accordance with FIG. 52. In another embodiment, Polymorph A exhibits an X-ray powder diffraction pattern obtained using Cu Kα radiation, having peaks with 2-theta values substantially in accordance with Table XIII.

Polymorph A of MC3 trimesate can also be defined according to its differential scanning calorimetry thermogram. In one embodiment, the polymorph exhibits a differential scanning calorimetry thermogram showing a primary endotherm expressed in units of ° C. at a temperature of 184+/−2° C. In one embodiment, the polymorph exhibits a differential scanning calorimetry thermogram showing a primary endotherm expressed in units of ° C. at a temperature of 186+/−2° C. and a second primary endotherm expressed in units of ° C. at a temperature of 90+/−2° C. In yet another embodiment, the polymorph exhibits a differential scanning calorimetry thermogram substantially in accordance with FIG. 53 or FIG. 54.

Polymorph B of MC3 trimesate can be defined according to its X-ray powder diffraction pattern. Accordingly, Polymorph B of MC3 trimesate exhibits an X-ray powder diffraction pattern obtained using Cu Kα radiation, having two, three, four or more characteristic peaks expressed in degrees 2-theta (+/−0.4) at 4.8, 5.4, 7.2, 9.7, 12.1, 14.5, 17.0, 19.4, 21.9, 24.3, 26.8, 29.3, or 31.8. In one embodiment, Polymorph B exhibits an X-ray powder diffraction pattern obtained using Cu Kα radiation, having peaks with 2-theta values substantially in accordance with FIG. 59. In another embodiment, Polymorph B exhibits an X-ray powder diffraction pattern obtained using Cu Kα radiation, having peaks with 2-theta values substantially in accordance with Table XIV.

Polymorph B of MC3 trimesate can also be defined according to its differential scanning calorimetry thermogram. In one embodiment, the polymorph exhibits a differential scanning calorimetry thermogram showing a primary endotherm expressed in units of ° C. at a temperature of 187+/−2° C. In another embodiment, the polymorph exhibits a differential scanning calorimetry thermogram substantially in accordance with FIG. 60.

Another aspect of the disclosure relates to the preparation of the salt or cocrystal of heptadecan-9-yl 8-((2-hydroxyethyl)amino)octanoate (“Compound 1”) and a compound selected from the group consisting of 4-hydroxybenzoic acid, oxalic acid, trimellitic acid, orotic acid, trimesic acid, and sulfuric acid.

Also provided herein is a method for preparing the salt or cocrystal of heptadecan-9-yl 8-((2-hydroxyethyl)(6-oxo-6-(undecyloxy)hexyl)amino)octanoate (“Compound 2”) and a compound selected from the group consisting of trimesic acid, (−)-2,3-dibenzoyl-L-tartaric acid, 4-acetamido benzoic acid, (+)-L-tartaric acid, and methanesulfonic acid.

This disclosure also provides a method of preparing the salt or cocrystal of heptadecan-9-yl 8-((2-hydroxyethyl)(8-(nonyloxy)-8-oxooctyl)amino)octanoate (“Compound 3”) and trimesic acid.

This disclosure also provides a method of preparing the salt or cocrystal of (6Z,9Z,28Z,31Z)-heptatriaconta-6,9,28,31-tetraen-19-yl 4-(dimethylamino)butanoate (“MC3”) (“MC3”) and trimesic acid.

In still another aspect, provided herein is a process of synthesizing Compound 2, Compound 3, or an analog thereof by reacting a salt or cocrystal of Compound 1 disclosed herein with a suitable electrophile, such as an ester substituted with a halogen (e.g., Br or I).

Also provided herein is a process of purifying Compound 1, 2, or 3 by forming a salt or cocrystal thereof disclosed herein to separate the salt or cocrystal thereof from the impurities. The method may further comprise neutralizing the salt or cocrystal to convert to Compound 1, 2, or 3 (i.e., a free base).

In one embodiment, the process of the present disclosure is advantageous as compared to other processes in that the process of the disclosure produces Compound 1, 2, or 3 or a salt or cocrystal thereof at a large scale and/or at a high purity, e.g., such that cumbersome purification (e.g., column chromatography, extraction, phase separation, distillation and solvent evaporation) is not needed. In one embodiment, the process of the present disclosure is able to process at least 100 g, 200 g, 500 g, or more (e.g., 1 kg, 2 kg, 5 kg, 10 kg, 20 kg, 50 kg, 100 kg, 200 kg, 500 kg, or 1000 kg or more) Compound 1, 2, or 3 or a salt or cocrystal thereof. In one embodiment, the process of the present disclosure is able to produce Compound 1, 2, or 3 or a salt or cocrystal thereof at least at a purity of at least 75%, 80%, 85%, 90%, 95%, 96%, 97%, 98%, 99%, or 99.5%, or higher. In one embodiment, the process of the present disclosure is able to produce Compound 1, 2, or 3 or a salt or cocrystal thereof with little or no impurity. In one embodiment, the impurity produced in the process of the present disclosure, even if produced, is easy to be separated from Compound 1, 2, or 3 or a salt or cocrystal thereof, without cumbersome purification (e.g., column chromatography, extraction, phase separation, distillation and solvent evaporation).

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present invention, suitable methods and materials are described below. In the case of conflict, the present specification, including definitions, will control. In addition, the materials, methods and examples are illustrative only and are not intended to be limiting.

Other features and advantages of the invention will be apparent from the following drawings, detailed description and claims.

### BRIEF DESCRIPTION OF THE DRAWINGS

FIG. 1 depicts a representative X-ray powder diffraction (XRPD) pattern overlay of heptadecan-9-yl 8-((2-hydroxyethyl)amino)octanoate 4-hydroxybenzoate Polymorph A batches, i.e., 100 mg and 10 mg batches or batches Nos. 1 and 2.

FIG. 2 depicts a ¹H NMR spectrum of heptadecan-9-yl 8-((2-hydroxyethyl)amino)octanoate 4-hydroxybenzoate Polymorph A, batch No. 2.

FIG. 3 depicts thermo-gravimetric analysis (TGA) and differential scanning calorimetry (DSC) data for heptadecan-9-yl 8-((2-hydroxyethyl)amino)octanoate 4-hydroxybenzoate Polymorph A, batch No. 2.

FIG. 4 depicts cyclic DSC data for heptadecan-9-yl 8-((2-hydroxyethyl)amino)octanoate 4-hydroxybenzoate Polymorph A, batch No. 2.

FIG. 5 depicts a representative XRPD pattern overlay of heptadecan-9-yl 8-((2-hydroxyethyl)amino)octanoate 4-hydroxybenzoate Polymorph A (i.e., Type A in the figure), batch No. 2, before and after heating.

FIG. 6 depicts TGA and DSC data for heptadecan-9-yl 8-((2-hydroxyethyl)amino)octanoate 4-hydroxybenzoate Polymorph A, batch No. 1.

FIG. 7 depicts variable temperature X-ray powder diffraction (VT-XRPD) pattern overlay of heptadecan-9-yl 8-((2-hydroxyethyl)amino)octanoate 4-hydroxybenzoate Polymorph A batch No. 1, before and after heating. Type A ref. in this figure is heptadecan-9-yl 8-((2-hydroxyethyl)amino)octanoate 4-hydroxybenzoate Polymorph A, batch No. 2.

FIG. 8 depicts dynamic vapor sorption (DVS) data at 25° C. for heptadecan-9-yl 8-((2-hydroxyethyl)amino)octanoate 4-hydroxybenzoate Polymorph A, batch No. 1.

FIG. 9 depicts an XRPD pattern overlay of heptadecan-9-yl 8-((2-hydroxyethyl)amino)octanoate 4-hydroxybenzoate Polymorph A, batch No. 1, before and after DVS.

FIG. 10 depicts a polarized light microscopy (PLM) image for heptadecan-9-yl 8-((2-hydroxyethyl)amino)octanoate 4-hydroxybenzoate Polymorph A, batch No. 1.

FIG. 11 depicts a representative XRPD pattern overlay of heptadecan-9-yl 8-((2-hydroxyethyl)amino)octanoate trimellitate Polymorph A batches, i.e., 100 mg and 10 mg batches or batches Nos. 1 and 2.

FIG. 12 depicts an ¹H NMR spectrum of heptadecan-9-yl 8-((2-hydroxyethyl)amino)octanoate trimellitate Polymorph A, batch No. 2.

FIG. 13 depicts TGA and DSC data for heptadecan-9-yl 8-((2-hydroxyethyl)amino)octanoate trimellitate Polymorph A, batch No. 1.

FIG. 14 depicts a VT-XRPD pattern overlay of heptadecan-9-yl 8-((2-hydroxyethyl)amino)octanoate trimellitate Polymorph A batch No. 1, before and after heating.

FIG. 15 depicts DVS data at 25° C. for heptadecan-9-yl 8-((2-hydroxyethyl)amino)octanoate trimellitate Polymorph A, batch No. 1.

FIG. 16 depicts an XRPD pattern overlay of heptadecan-9-yl 8-((2-hydroxyethyl)amino)octanoate trimellitate Polymorph A, batch No. 1, before and after DVS.

FIG. 17 depicts a polarized light microscopy (PLM) image for heptadecan-9-yl 8-((2-hydroxyethyl)amino)octanoate trimellitate Polymorph A, batch No. 1.

FIG. 18 depicts a representative XRPD pattern overlay of heptadecan-9-yl 8-((2-hydroxyethyl)amino)octanoate orotate Polymorphs A and B.

FIG. 19 depicts an ¹H NMR spectrum of heptadecan-9-yl 8-((2-hydroxyethyl)amino)octanoate orotate Polymorph A.

FIG. 20 depicts TGA and DSC data for heptadecan-9-yl 8-((2-hydroxyethyl)amino)octanoate orotate Polymorph A.

FIG. 21 depicts a VT-XRPD pattern overlay of heptadecan-9-yl 8-((2-hydroxyethyl)amino)octanoate orotate Polymorph A, before and after heating.

FIG. 22 depicts heating-cooling DSC curve for heptadecan-9-yl 8-((2-hydroxyethyl)amino)octanoate orotate Polymorph A.

FIG. 23 depicts TGA and DSC data for heptadecan-9-yl 8-((2-hydroxyethyl)amino)octanoate orotate Polymorphs B.

FIG. 24 depicts cyclic DSC data for heptadecan-9-yl 8-((2-hydroxyethyl)amino)octanoate orotate Polymorphs B.

FIG. 25 depicts an XRPD pattern overlay of heptadecan-9-yl 8-((2-hydroxyethyl)amino)octanoate orotate Polymorph B, before and after cyclic DSC.

FIG. 26 depicts DVS data at 25° C. for heptadecan-9-yl 8-((2-hydroxyethyl)amino)octanoate orotate Polymorphs B.

FIG. 27 depicts an XRPD pattern overlay of heptadecan-9-yl 8-((2-hydroxyethyl)amino)octanoate orotate Polymorph B, before and after DVS.

FIG. 28 depicts a PLM image of heptadecan-9-yl 8-((2-hydroxyethyl)amino)octanoate orotate Polymorph B.

FIG. 29 depicts a PLM image of heptadecan-9-yl 8-((2-hydroxyethyl)amino)octanoate sulfate Polymorph A.

FIG. 30 depicts an XRPD pattern of heptadecan-9-yl 8-((2-hydroxyethyl)amino)octanoate sulfate Polymorph A.

FIG. 31 depicts TGA and DSC data of heptadecan-9-yl 8-((2-hydroxyethyl)amino)octanoate sulfate Polymorph A.

FIG. 32 depicts an XRPD pattern of heptadecan-9-yl 8-((2-hydroxyethyl)amino)octanoate trimesate Polymorph A.

FIG. 33 depicts an ¹H NMR overlay of heptadecan-9-yl 8-((2-hydroxyethyl)amino)octanoate trimesate and freebase.

FIG. 34 depicts TGA data of heptadecan-9-yl 8-((2-hydroxyethyl)amino)octanoate.

FIG. 35 depicts cyclic DSC data of heptadecan-9-yl 8-((2-hydroxyethyl)amino)octanoate (heating/cooling rate: 10° C./min).

FIG. 36 depicts an XRPD pattern overlay of heptadecan-9-yl 8-((2-hydroxyethyl)(6-oxo-6-(undecyloxy)hexyl)amino)octanoate dibenzoyl-L-tartrate Polymorph A and the corresponding acid, dibenzoyl-L-tartaric acid.

FIG. 37 depicts TGA and DSC data for heptadecan-9-yl 8-((2-hydroxyethyl)(6-oxo-6-(undecyloxy)hexyl)amino)octanoate dibenzoyl-L-tartrate Polymorph A.

FIG. 38 depicts an XRPD pattern overlay of heptadecan-9-yl 8-((2-hydroxyethyl)(6-oxo-6-(undecyloxy)hexyl)amino)octanoate trimesate Polymorph A and the corresponding acid, trimesic acid.

FIG. 39 depicts TGA and DSC data for heptadecan-9-yl 8-((2-hydroxyethyl)(6-oxo-6-(undecyloxy)hexyl)amino)octanoate trimesate Polymorph A.

FIG. 40 depicts an XRPD pattern overlay of heptadecan-9-yl 8-((2-hydroxyethyl)(6-oxo-6-(undecyloxy)hexyl)amino)octanoate L-tartrate Polymorph A and the corresponding acid, L-tartaric acid.

FIG. 41 depicts TGA and DSC data for heptadecan-9-yl 8-((2-hydroxyethyl)(6-oxo-6-(undecyloxy)hexyl)amino)octanoate L-tartrate Polymorph A.

FIG. 42 depicts an XRPD pattern of heptadecan-9-yl 8-((2-hydroxyethyl)(6-oxo-6-(undecyloxy)hexyl)amino)octanoate mesylate Polymorph A.

FIG. 43 depicts TGA and DSC data for heptadecan-9-yl 8-((2-hydroxyethyl)(6-oxo-6-(undecyloxy)hexyl)amino)octanoate mesylate Polymorph A.

FIG. 44 depicts an XRPD pattern overlay of heptadecan-9-yl 8-((2-hydroxyethyl)(6-oxo-6-(undecyloxy)hexyl)amino)octanoate 4-acetamido benzoate Polymorph A and the corresponding acid, 4-acetamido benzoic acid.

FIG. 45 depicts TGA and DSC data for heptadecan-9-yl 8-((2-hydroxyethyl)(6-oxo-6-(undecyloxy)hexyl)amino)octanoate 4-acetamido benzoate Polymorph A.

FIG. 46 depicts an XRPD pattern overlay of heptadecan-9-yl 8-((2-hydroxyethyl)(8-(nonyloxy)-8-oxooctyl)amino)octanoate trimesate Polymorph A and the corresponding acid, trimesic acid.

FIG. 47 depicts TGA and DSC data for heptadecan-9-yl 8-((2-hydroxyethyl)(8-(nonyloxy)-8-oxooctyl)amino)octanoate trimesate Polymorph A.

FIG. 48 depicts an XRPD pattern of heptadecan-9-yl 8-((2-hydroxyethyl)(8-(nonyloxy)-8-oxooctyl)amino)octanoate trimesate Polymorph B.

FIG. 49 depicts TGA and DSC data for heptadecan-9-yl 8-((2-hydroxyethyl)(8-(nonyloxy)-8-oxooctyl)amino)octanoate trimesate Polymorph B.

FIG. 50 depicts an ¹H NMR overlay of heptadecan-9-yl 8-((2-hydroxyethyl)(8-(nonyloxy)-8-oxooctyl)amino)octanoate trimesate Polymorph B and freebase.

FIG. 51 is a polarized light microscopy (PLM) image of heptadecan-9-yl 8-((2-hydroxyethyl)(8-(nonyloxy)-8-oxooctyl)amino)octanoate trimesate Polymorph B.

FIG. 52 is an XRPD pattern of (6Z,9Z,28Z,31Z)-heptatriaconta-6,9,28,31-tetraen-19-yl 4-(dimethylamino)butanoate trimesate Type A polymorph.

FIG. 53 depicts TGA and DSC data for (6Z,9Z,28Z,31Z)-heptatriaconta-6,9,28,31-tetraen-19-yl 4-(dimethylamino)butanoate trimesate Type A polymorph prepared with cyclohexane.

FIG. 54 depicts TGA and DSC data for (6Z,9Z,28Z,31Z)-heptatriaconta-6,9,28,31-tetraen-19-yl 4-(dimethylamino)butanoate trimesate Type A polymorph prepared with EtOAc.

FIG. 55 is a polarized light microscopy (PLM) image of (6Z,9Z,28Z,31Z)-heptatriaconta-6,9,28,31-tetraen-19-yl 4-(dimethylamino)butanoate trimesate Type A polymorph prepared with cyclohexane.

FIG. 56 is a polarized light microscopy (PLM) image of (6Z,9Z,28Z,31Z)-heptatriaconta-6,9,28,31-tetraen-19-yl 4-(dimethylamino)butanoate trimesate Type A polymorph prepared with EtOAc.

FIG. 57 depicts DVS data at 25° C. for (6Z,9Z,28Z,31Z)-heptatriaconta-6,9,28,31-tetraen-19-yl 4-(dimethylamino)butanoate trimesate Type A polymorphs before and after DVS.

FIG. 58 is an XRPD pattern overlay of (6Z,9Z,28Z,31Z)-heptatriaconta-6,9,28,31-tetraen-19-yl 4-(dimethylamino)butanoate trimesate Type A polymorphs before and after DVS.

FIG. 59 is an XRPD pattern of (6Z,9Z,28Z,31Z)-heptatriaconta-6,9,28,31-tetraen-19-yl 4-(dimethylamino)butanoate trimesate Type B polymorph.

FIG. 60 depicts TGA and DSC data for (6Z,9Z,28Z,31Z)-heptatriaconta-6,9,28,31-tetraen-19-yl 4-(dimethylamino)butanoate trimesate Type B.

FIG. 61 is a polarized light microscopy (PLM) image of (6Z,9Z,28Z,31Z)-heptatriaconta-6,9,28,31-tetraen-19-yl 4-(dimethylamino)butanoate trimesate Type B polymorph.

### DETAILED DESCRIPTION

The solid form (e.g., crystal state) of a compound may be important when the compound is used for pharmaceutical purposes. Compared with an amorphous solid or viscous oil, the physical properties of a crystalline compound are generally enhanced. These properties change from one solid form to another, which may impact its suitability for pharmaceutical use. In addition, different solid forms of a crystalline compound may incorporate different types and/or different amounts of impurities. Different solid forms of a compound may also have different chemical stability upon exposure to heat, light and/or moisture (e.g., atmospheric moisture) over a period of time, or different rates of dissolution. Long-chain amino lipids are usually oils at room temperature. Solid forms of these lipids are desirable for e.g., improving handling, improving stability (such as storage stability), simplifying purification process, simplifying large-scale production process and/or increasing accuracy in measurements and characterization of lipids.

Provided herein are novel solid forms (e.g., crystalline forms) of each of Compound 1, Compound 2, and Compound 3, the structure of each of which is provided below:

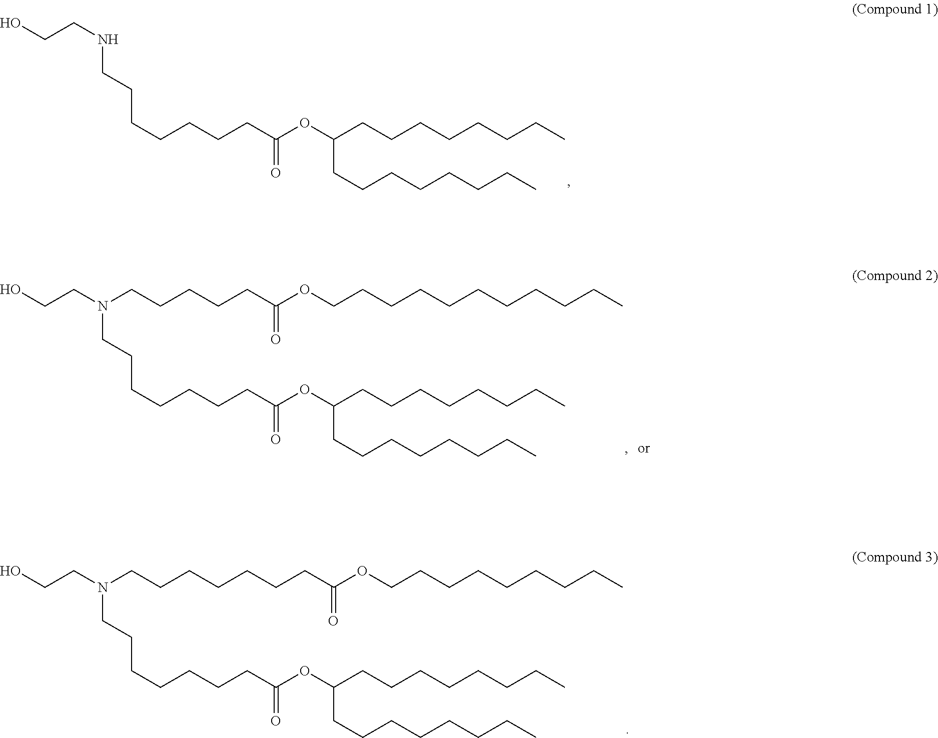 [SMILES: CCCCCCCCC(CCCCCCCC)OC(=O)CCCCCCCNCCO ; CCCCCCCCCCCOC(=O)CCCCCN(CCO)CCCCCCCC(=O)OC(CCCCCCCC)CCCCCCCC ; CCCCCCCCCOC(=O)CCCCCCCN(CCO)CCCCCCCC(=O)OC(CCCCCCCC)CCCCCCCC]

In another aspect, provided herein are novel solid forms (e.g., crystalline forms) of (6Z,9Z,28Z,31Z)-heptatriaconta-6,9,28,31-tetraen-19-yl 4-(dimethylamino)butanoate (“MC3”), the structure of which is provided below:

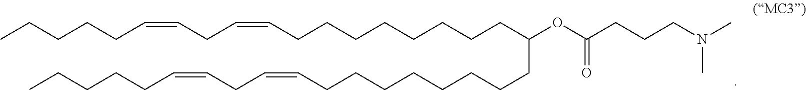 [SMILES: CCCCC/C=C\C/C=C\CCCCCCCCC(CCCCCCCC/C=C\C/C=C\CCCCC)OC(=O)CCCN(C)C]

In one aspect, disclosed herein is salt or cocrystal of Compound 1, 2, or 3, which has a melting point of about 50° C. or greater (e.g., about 60° C., about 70° C. or greater). For example, the salt or cocrystal of Compound 1, 2, or 3 is formed between Compound 1, 2, or 3 and a coformer compound (e.g., an acid). In another aspect, the salt or cocrystal of Compound 3 has a melting point of about 270° C. or greater (e.g., about 280° C., about 290° C. or greater).

As used herein, “Compound 1” refers to heptadecan-9-yl 8-((2-hydroxyethyl)amino)octanoate; “Compound 2” refers to heptadecan-9-yl 8-((2-hydroxyethyl)(6-oxo-6-(undecyloxy)hexyl)amino)octanoate; and “Compound 3” refers to heptadecan-9-yl 8-((2-hydroxyethyl)(8-(nonyloxy)-8-oxooctyl)amino)octanoate. Compound 1 can be used as a starting material for the synthesis of Compound 2 or 3.

As used herein, “MC 3” refers to (6Z,9Z,28Z,31Z)-heptatriaconta-6,9,28,31-tetraen-19-yl 4-(dimethylamino)butanoate.

In one aspect, this disclosure is directed to a salt or cocrystal of Compound 1 and a compound selected from the group consisting of 4-hydroxybenzoic acid, oxalic acid, trimellitic acid, orotic acid, trimesic acid, and sulfuric acid. For example, the compound is 4-hydroxybenzoic acid. For example, the compound is oxalic acid.

Also described herein are polymorphic forms of a salt or cocrystal of Compound 1, e.g., Polymorphs A and B of 4-hydroxybenzoate of Compound 1, or Polymorphs A and B of orotate of Compound 1.

In one aspect, this disclosure is directed to a salt or cocrystal of (6Z,9Z,28Z,31Z)-heptatriaconta-6,9,28,31-tetraen-19-yl 4-(dimethylamino)butanoate (“MC3”). In another aspect, the salt or cocrystal of MC3 has a melting point of about 150° C. or greater (e.g., about 160° C., about 170° C., about 180° C. or greater, about 190° C. or greater). In another aspect, disclosed herein is a salt or cocrystal of (6Z,9Z,28Z,31Z)-heptatriaconta-6,9,28,31-tetraen-19-yl 4-(dimethylamino)butanoate (“MC3”). In another aspect, the salt or cocrystal of MC3 has a melting point of about 50° C. or greater (e.g., about 60° C., about 70° C., about 80° C. or greater). For example, the salt or cocrystal of MC3 is formed between MC3 and a coformer compound (e.g., an acid).

The ability of a substance to exist in more than one crystal form is defined as polymorphism; the different crystal forms of a particular substance are referred to as “polymorphs” of one another. In general, polymorphism is affected by the ability of a molecule of a substance (or its salt, cocrystal, or hydrate) to change its conformation or to form different intermolecular or intra-molecular interactions, (e.g., different hydrogen bond configurations), which is reflected in different atomic arrangements in the crystal lattices of different polymorphs. In contrast, the overall external form of a substance is known as “morphology,” which refers to the external shape of the crystal and the planes present, without reference to the internal structure. A particular crystalline polymorph can display different morphology based on different conditions, such as, for example, growth rate, stirring, and the presence of impurities.

The different polymorphs of a substance may possess different energies of the crystal lattice and, thus, in solid state they can show different physical properties such as form, density, melting point, color, stability, solubility, dissolution rate, etc., which can, in turn, effect the stability, dissolution rate and/or bioavailability of a given polymorph and its suitability for use as a pharmaceutical and in pharmaceutical compositions.

Polymorph A of 4-hydroxybenzoate of Compound 1 has a number of advantageous physical properties over its free base form, as well as other salts of the free base. In particular, Polymorph A of 4-hydroxybenzoate of Compound 1 has low hygroscopicity compared to other salt forms of Compound 1. More particularly, Polymorph A of 4-hydroxybenzoate of Compound 1 has low hygroscopicity compared to Polymorph A of Compound 1 trimellitate and Polymorph B of Compound 1 orotate (see, e.g., Table 1-2). Crystal forms that are highly hygroscopic may also be unstable, as the compound's dissolution rate (and other physico-chemical properties) may change as it is stored in settings with varying humidity. Also, hygroscopicity can impact large-scale handling and manufacturing of a compound, as it can be difficult to determine the true weight of a hygroscopic agent when using it for reactions or when preparing a pharmaceutical composition comprising that agent. For example, in large scale medicinal formulating preparations, highly hygroscopic compounds can result in batch manufacturing inconsistency creating clinical and/or prescribing difficulties. For example, when Compound 1 is used as a starting material for the synthesis of Compound 2 or 3, Polymorph A of 4-hydroxybenzoate of Compound 1 has a low hygoscopicity compared to other salt forms of Compound 1, and as such, it may be stored over appreciable periods or conditions (e.g., relative humidity conditions), and not suffer from weight changes that would be detrimental for consistent production of Compound 2 or 3.

In certain embodiments, Polymorph A of 4-hydroxybenzoate of Compound 1 is identifiable on the basis of characteristic peaks in an X-ray powder diffraction analysis. X-ray powder diffraction pattern, also referred to as XRPD pattern, is a scientific technique involving the scattering of x-rays by crystal atoms, producing a diffraction pattern that yields information about the structure of the crystal. In certain embodiments, Polymorph A of 4-hydroxybenzoate of Compound 1 exhibits an X-ray powder diffraction (XRPD) pattern obtained using Cu Kα radiation, having from two (2) to seven (7) characteristic peaks expressed in degrees 2-theta at 4.5, 6.8, 9.1, 11.4, 13.7, 18.3, 20.1, and 20.6.

The skilled artisan recognizes that some variation is associated with 2-theta measurements in XRPD. Typically, 2-theta values may vary from ±0.1 to ±0.2. Such slight variation can be caused, for example, by sample preparation, instrument configurations and other experimental factors. The skilled artisan appreciates that such variation in values are greatest with low 2-theta values, and least with high 2-theta values. The skilled artisan recognizes that different instruments may provide substantially the same XRPD pattern, even though the 2-theta values vary slightly. Moreover, the skilled artisan appreciates that the same instrument may provide substantially the same XRPD pattern for the same or different samples even though the XRPD of the respectively collected XRPD patterns vary slightly in the 2-theta values.

The skilled artisan also appreciates that XRPD patterns of the same sample (taken on the same or different instruments) may exhibit variations in peak intensity at the different 2-theta values. The skilled artisan also appreciates that XRPD patterns of different samples of the same polymorph (taken on the same or different instruments) may also exhibit variations in peak intensity at the different 2-theta values. XRPD patterns can be substantially the same pattern even though they have corresponding 2-theta signals that vary in their peak intensities.

In one embodiment, Polymorph A of 4-hydroxybenzoate of Compound 1 exhibits an X-ray powder diffraction pattern obtained using Cu Kα radiation, having two or more characteristic peaks expressed in degrees 2-theta (+/−0.2) selected from the group consisting of 4.5, 6.8, 9.1, and 11.4. In another embodiment, Polymorph A of 4-hydroxybenzoate of Compound 1 exhibits an X-ray powder diffraction pattern obtained using Cu Kα radiation, having three or more characteristic peaks expressed in degrees 2-theta (+/−0.2) selected from the group consisting of 4.5, 6.8, 9.1, 11.4, and 13.7. In another embodiment, Polymorph A of 4-hydroxybenzoate of Compound 1 exhibits an X-ray powder diffraction pattern obtained using Cu Kα radiation, having four or more characteristic peaks expressed in degrees 2-theta (+/−0.2) selected from the group consisting of 4.5, 6.8, 9.1, 11.4, and 13.7. In another embodiment, Polymorph A of 4-hydroxybenzoate of Compound 1 exhibits an X-ray powder diffraction pattern obtained using Cu Kα radiation, having characteristic peaks expressed in degrees 2-theta (+/−0.2) at 4.5, 6.8, 9.1, 11.4, 13.7, 18.3, 20.1, and 20.6. In one embodiment, Polymorph A of 4-hydroxybenzoate of Compound 1 exhibits an X-ray powder diffraction pattern obtained using Cu Kα radiation, having two, three, four, or more characteristic peaks expressed in degrees 2-theta (+/−0.2) selected from the group consisting of 4.5, 6.8, 9.1, 11.4, and 13.7.

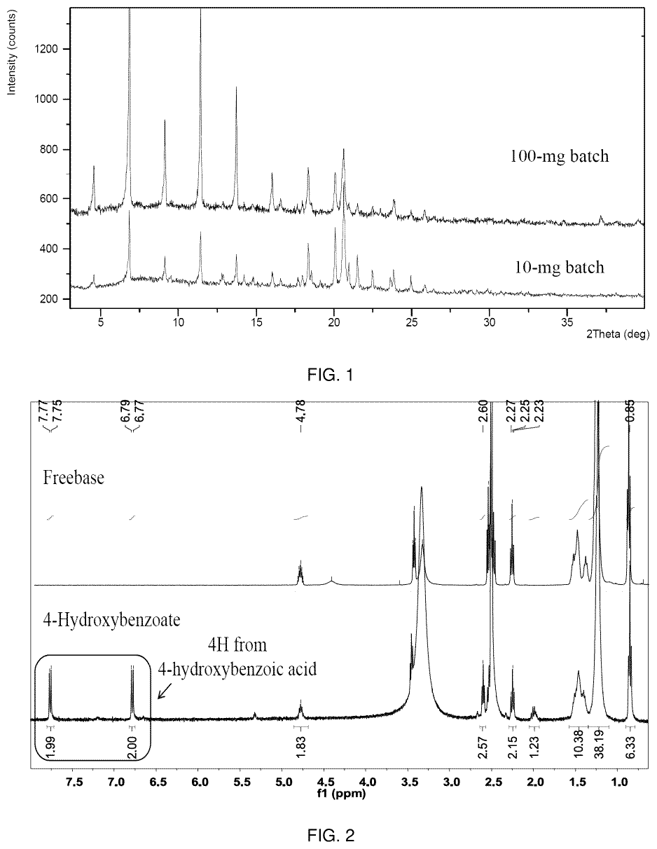

In a particular embodiment, Polymorph A of 4-hydroxybenzoate of Compound 1 exhibits an X-ray powder diffraction pattern obtained using Cu Kα radiation, having at least eight characteristic peaks expressed in degrees 2-theta (+/−0.2), selected from the group consisting of 4.5, 6.8, 9.1, 11.4, 13.7, 16.0, 18.3, 20.1, and 20.6. In another particular embodiment, Polymorph A of 4-hydroxybenzoate of Compound 1 exhibits an X-ray powder diffraction pattern obtained using Cu Kα radiation, having at least nine characteristic peaks expressed in degrees 2-theta (+/−0.2), selected from the group consisting of 4.5, 6.8, 9.1, 11.4, 13.7, 16.0, 16.6, 18.3, 20.1, and 20.6. In a further embodiment, Polymorph A of 4-hydroxybenzoate of Compound 1 exhibits an X-ray powder diffraction pattern obtained using Cu Kα radiation, having at least ten characteristic peaks expressed in degrees 2-theta (+/−0.2), selected from the group consisting of 4.5, 6.8, 9.1, 11.4, 13.7, 16.0, 16.6, 18.3, 20.1, 20.6, and 21.5. In one embodiment, Polymorph A exhibits an X-ray powder diffraction pattern obtained using Cu Kα radiation, having peaks with 2-theta values substantially in accordance with FIG. 1. In another embodiment, Polymorph A exhibits an X-ray powder diffraction pattern obtained using Cu Kα radiation, having peaks with 2-theta values substantially in accordance with Table I below.

TABLE I

| Peak | Position [°2Th.] |
|---|---|
| 1. | 4.5 |
| 2. | 6.8 |
| 3. | 9.1 |
| 4. | 11.4 |
| 5. | 13.7 |
| 6. | 16.0 |
| 7. | 16.6 |
| 8. | 18.3 |
| 9. | 20.1 |
| 10. | 20.6 |
| 11. | 21.5 |
| 12. | 23.8 |
| 13. | 24.9 |
| 14. | 25.8 |

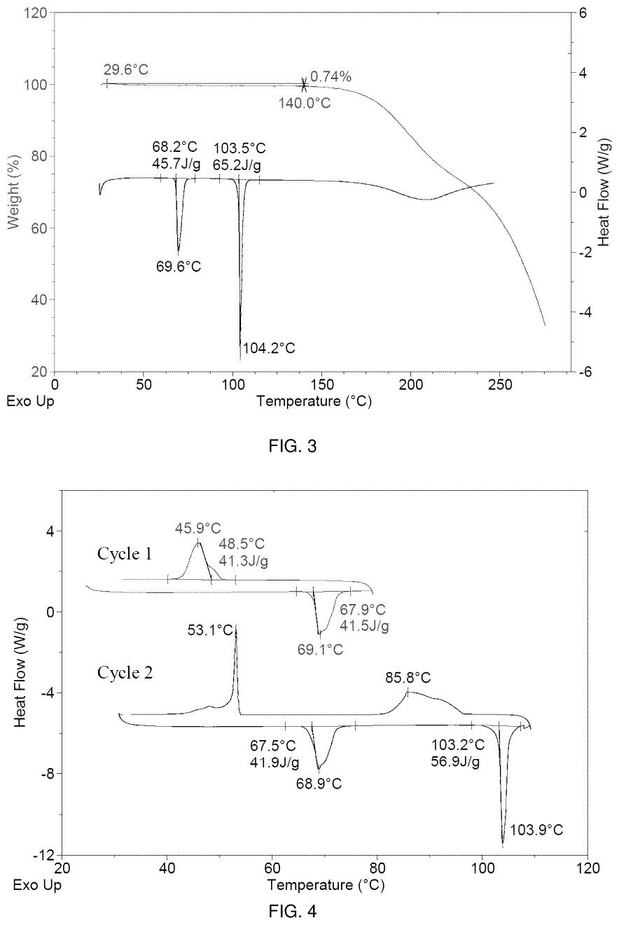

In other embodiments, Polymorph A of 4-hydroxybenzoate of Compound 1 is identifiable on the basis of a characteristic peak observed in a differential scanning calorimetry thermogram. Differential scanning calorimetry, or DSC, is a thermoanalytical technique in which the difference in the amount of heat required to increase the temperature of a sample and reference is measured as a function of temperature. In one embodiment, Polymorph A of 4-hydroxybenzoate of Compound 1 exhibits a differential scanning calorimetry thermogram showing a characteristic primary endotherm peak expressed in units of ° C. with an onset temperature of about 103+/−2° C. In another embodiment, Polymorph A of 4-hydroxybenzoate of Compound 1 exhibits a differential scanning calorimetry thermogram showing a characteristic second primary endotherm expressed in units of ° C. with an onset temperature of about 68+/−2° C. In another embodiment, Polymorph A of 4-hydroxybenzoate of Compound 1 exhibits a differential scanning calorimetry thermogram substantially in accordance with the lower curve shown in FIG. 3.

In another embodiment, provided herein is Polymorph A of 4-hydroxybenzoate of Compound 1, wherein the solid form undergoes a weight increase of less than 1.5% (e.g., less than 1%, or less than 0.6%) upon increasing relative humidity from 5.0% to 95.0% at e.g., 25° C. In another embodiment, Polymorph A of 4-hydroxybenzoate of Compound 1 is characterized as having a dynamic vapor sorption profile that is substantially in accordance with FIG. 8.

In one embodiment, Polymorph A of 4-hydroxybenzoate of Compound 1 is substantially free of impurities, meaning there is not a significant amount of impurities present in the sample of Polymorph A. In another embodiment, Polymorph A is a crystalline solid substantially free of amorphous Compound 1 (or any of its amorphous salt forms). In yet another embodiment, Polymorph A is a crystalline solid substantially free of other polymorphs of 4-hydroxybenzoate of Compound 1 and substantially free of amorphous Compound 1 (or any of its amorphous salt forms). For example, Polymorph A is a crystalline solid substantially free of Polymorph B of 4-hydroxybenzoate of Compound 1 and substantially free of amorphous Compound 1 (or any of its amorphous salt forms). The skilled artisan understands that a solid sample of Polymorph A may also include other polymorphs (e.g., Polymorph A), and/or amorphous Compound 1 (or any of its amorphous salt forms)

As used herein, the term “substantially free of amorphous Compound 1” means that the compound contains no significant amount of amorphous Compound 1 (or any of its amorphous salt forms). In another embodiment, a sample of a salt or cocrystal of Compound 1 comprises Polymorph A of 4-hydroxybenzoate of Compound 1 substantially free of other polymorphs (e.g., Polymorph B of 4-hydroxybenzoate of Compound 1). As used herein, the term “substantially free of other polymorphs” means that a sample of crystalline Compound 1 4-hydroxybenzoate contains no significant amount of other polymorphs (e.g., Polymorph B). In certain embodiments, at least about 90% by weight of a sample is Polymorph A, with only 10% being other polymorphs (e.g., Polymorph B) and/or amorphous Compound 1 (or any of its amorphous salt forms). In certain embodiments, at least about 95% by weight of a sample is Polymorph A, with only 5% being other polymorphs (e.g., Polymorph B) and/or amorphous Compound 1 (or any of its amorphous salt forms). In still other embodiments, at least about 98% by weight of a sample is Polymorph A, with only 2% by weight being other polymorphs (e.g., Polymorph B) and/or amorphous Compound 1 (or any of its amorphous salt forms). In still other embodiments, at least about 99% by weight of a sample is Polymorph A, with only 1% by weight being other polymorphs (e.g., Polymorph B) and/or amorphous Compound 1 (or any of its amorphous salt forms). In still other embodiments, at least about 99.5% by weight of a sample is Polymorph A, with only 0.5% by weight being other polymorphs (e.g., Polymorph B) and/or amorphous Compound 1 (or any of its amorphous salt forms). In still other embodiments, at least about 99.9% by weight of a sample is Polymorph A, with only 0.1% by weight being other polymorphs (e.g., Polymorph B) and/or amorphous Compound 1 (or any of its amorphous salt forms).

In certain embodiments, a sample of a salt or cocrystal of Compound 1 (e.g., Compound 1 oxalate or 4-hydroxybenzoate) may contain impurities. Non-limiting examples of impurities include other polymorph forms, or residual organic and inorganic molecules such as related impurities (e.g., intermediates used to make Compound 1 or by-products, e.g., heptadecan-9-yl 8-bromooctanoate and di(heptadecan-9-yl) 8,8′-((2-hydroxyethyl)azanediyl)dioctanoate), solvents, water or salts. In one embodiment, a sample of a salt or cocrystal of Compound 1, e.g., oxalate or 4-hydroxybenzoate Polymorph A is substantially free from impurities, meaning that no significant amount of impurities are present. In another embodiment, a sample of the salt or cocrystal of Compound 1 contains less than 10% weight by weight (wt/wt) total impurities. In another embodiment, a sample of the salt or cocrystal of Compound 1 contains less than 5% wt/wt total impurities. In another embodiment, a sample of the salt or cocrystal of Compound 1 contains less than 2% wt/wt total impurities. In another embodiment, a sample of the salt or cocrystal of Compound 1 contains less than 1% wt/wt total impurities. In yet another embodiment, a sample of the salt or cocrystal of Compound 1 contains less than 0.1% wt/wt total impurities. In yet another embodiment, a sample of the salt or cocrystal of Compound 1 does not contain a detectable amount of impurities.

Also disclosed herein are Polymorphs A and B of Compound 1 orotate. In a particular embodiment, Polymorph A of Compound 1 orotate exhibits an X-ray powder diffraction pattern obtained using Cu Kα radiation, having two, three, four, or more characteristic peaks expressed in degrees 2-theta (+/−0.2) selected from the group consisting of 5.3, 10.7, 13.3, 16.1, and 18.7. In one embodiment, Polymorph A exhibits an X-ray powder diffraction pattern obtained using Cu Kα radiation, having peaks with 2-theta values substantially in accordance with FIG. 18, lower profile. In another embodiment, Polymorph A exhibits an X-ray powder diffraction pattern obtained using Cu Kα radiation, having peaks with 2-theta values substantially in accordance with Table II below.

TABLE II

| Peak | Position [°2Th.] |
|---|---|
| 1. | 5.3 |
| 2. | 10.7 |
| 3. | 13.3 |
| 4. | 16.1 |
| 5. | 18.7 |
| 6. | 24.3 |
| 7. | 26.9 |

Polymorph B of Compound 1 orotate can be defined according to its X-ray powder diffraction pattern. Accordingly, in one embodiment, Polymorph B exhibits an X-ray powder diffraction pattern obtained using Cu Kα radiation, having two, three, four, or more characteristic peaks expressed in degrees 2-theta (+/−0.2) selected from the group consisting of 5.1, 7.5, 10.1, 12.7, 15.2, and 17.8. In one embodiment, Polymorph B exhibits an X-ray powder diffraction pattern obtained using Cu Kα radiation, having peaks with 2-theta values substantially in accordance with FIG. 18, upper profile. In another embodiment, Polymorph B exhibits an X-ray powder diffraction pattern obtained using Cu Kα radiation, having peaks with 2-theta values substantially in accordance with Table III.

TABLE III

| Peak | Position [°2Th.] |
|---|---|
| 1. | 5.1 |
| 2. | 7.5 |
| 3. | 10.1 |
| 4. | 12.7 |
| 5. | 15.2 |
| 6. | 17.8 |
| 7. | 20.2 |
| 8. | 25.5 |
| 9. | 28.2 |

In yet another embodiment, this disclosure provides Polymorph A of Compound 1 trimesate. In a particular embodiment, Polymorph A of Compound 1 trimesate exhibits an X-ray powder diffraction pattern obtained using Cu Kα radiation, having two, three, four, or more characteristic peaks expressed in degrees 2-theta (+/−0.2) selected from the group consisting of 3.3, 5.3, 6.7, 7.9, 10.5, 18.5, 21.3, 23.9, and 26.5. In one embodiment, Polymorph A exhibits an X-ray powder diffraction pattern obtained using Cu Kα radiation, having peaks with 2-theta values substantially in accordance with FIG. 32. In another embodiment, Polymorph A exhibits an X-ray powder diffraction pattern obtained using Cu Kα radiation, having peaks with 2-theta values substantially in accordance with Table IV below.

TABLE IV

| Peak | Position [°2Th.] |
|---|---|
| 1. | 3.3 |
| 2. | 5.3 |
| 3. | 6.7 |
| 4. | 7.9 |
| 5. | 10.5 |
| 6. | 13.6 |
| 7. | 18.5 |
| 8. | 21.3 |
| 9. | 23.9 |
| 10. | 26.5 |
| 11. | 29.1 |

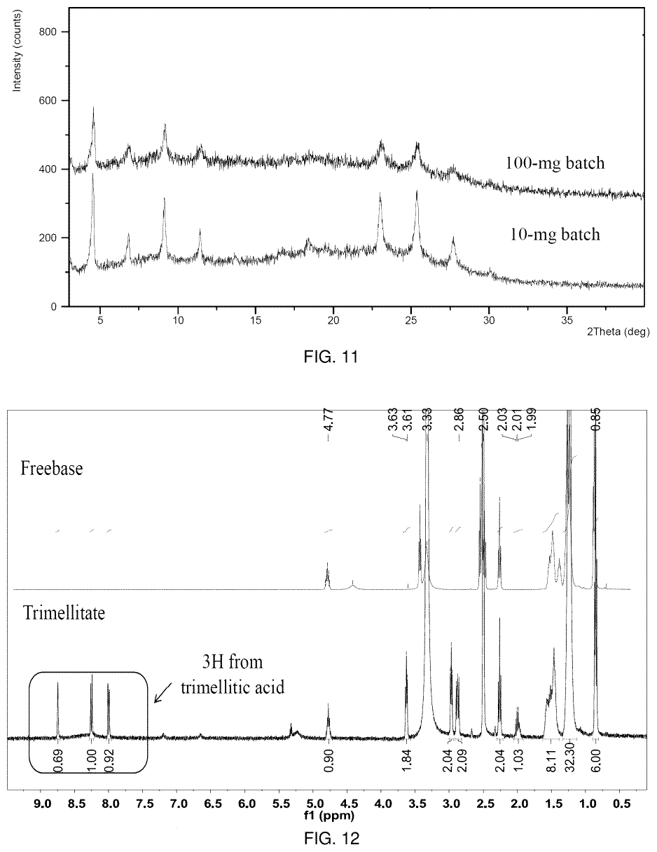

This disclosure also provides Polymorph A of Compound 1 trimellitate. In a particular embodiment, Polymorph A of Compound 1 trimellitate exhibits an X-ray powder diffraction pattern obtained using Cu Kα radiation, having two, three, four, or more characteristic peaks expressed in degrees 2-theta (+/−0.2) selected from the group consisting of 4.6, 6.8, 9.2, 11.5, 23.1, and 25.4. In one embodiment, Polymorph A exhibits an X-ray powder diffraction pattern obtained using Cu Kα radiation, having peaks with 2-theta values substantially in accordance with FIG. 11. In another embodiment, Polymorph A exhibits an X-ray powder diffraction pattern obtained using Cu Kα radiation, having peaks with 2-theta values substantially in accordance with Table V below.

TABLE V

| Peak | Position [°2Th.] |
|---|---|
| 1. | 4.6 |
| 2. | 6.8 |
| 3. | 9.2 |
| 4. | 11.5 |
| 5. | 23.1 |
| 6. | 25.4 |
| 7. | 27.7 |

Also provided herein is Polymorph A of Compound 1 sulfate. In a particular embodiment, Polymorph A of Compound 1 sulfate exhibits an X-ray powder diffraction pattern obtained using Cu Kα radiation, having two, three, four, or more characteristic peaks expressed in degrees 2-theta (+/−0.2) selected from the group consisting of 4.0, 11.8, 21.4, 21.8, and 22.8. In one embodiment, Polymorph A exhibits an X-ray powder diffraction pattern obtained using Cu Kα radiation, having peaks with 2-theta values substantially in accordance with FIG. 30. In another embodiment, Polymorph A exhibits an X-ray powder diffraction pattern obtained using Cu Kα radiation, having peaks with 2-theta values substantially in accordance with Table VI below.

TABLE VI

| Peak | Position [°2Th.] |
|---|---|
| 1. | 4.0 |
| 2. | 11.4 |
| 3. | 11.8 |
| 4. | 19.8 |
| 5. | 21.4 |
| 6. | 21.8 |
| 7. | 22.8 |

In another aspect, this disclosure is directed to a salt or cocrystal of heptadecan-9-yl 8-((2-hydroxyethyl)(6-oxo-6-(undecyloxy)hexyl)amino)octanoate (“Compound 2”) and a compound selected from the group consisting of trimesic acid, (−)-2,3-dibenzoyl-L-tartaric acid, 4-acetamido benzoic acid, (+)-L-tartaric acid, and methanesulfonic acid.

In one embodiment, this disclosure also provides Polymorph A of Compound 2 trimesate. In a particular embodiment, Polymorph A of Compound 2 trimesate exhibits an X-ray powder diffraction pattern obtained using Cu Kα radiation, having two, three, four, or more characteristic peaks expressed in degrees 2-theta (+/−0.2) selected from the group consisting of 3.4, 6.8, 10.2, 20.5, and 23.8. In one embodiment, Polymorph A exhibits an X-ray powder diffraction pattern obtained using Cu Kα radiation, having peaks with 2-theta values substantially in accordance with FIG. 38. In another embodiment, Polymorph A exhibits an X-ray powder diffraction pattern obtained using Cu Kα radiation, having peaks with 2-theta values substantially in accordance with Table VII below.

TABLE VII

| Peak | Position [°2Th.] |
|---|---|
| 1. | 3.4 |
| 2. | 6.8 |
| 3. | 10.2 |
| 4. | 20.5 |
| 5. | 23.8 |

In another embodiment, this disclosure also provides Polymorph A of Compound 2 dibenzoyl-L-tartrate. In a particular embodiment, Polymorph A of Compound 2 dibenzoyl-L-tartrate exhibits an X-ray powder diffraction pattern obtained using Cu Kα radiation, having two characteristic peaks expressed in degrees 2-theta (+/−0.2) at 6.1 and 9.1. In one embodiment, Polymorph A exhibits an X-ray powder diffraction pattern obtained using Cu Kα radiation, having peaks with 2-theta values substantially in accordance with FIG. 36, upper profile. In another embodiment, Polymorph A exhibits an X-ray powder diffraction pattern obtained using Cu Kα radiation, having peaks with 2-theta values substantially in accordance with Table VIII below.

TABLE VIII

| Peak | Pos. [°2Th.] |
|---|---|
| 1 | 6.1 |
| 2 | 9.1 |

In yet another embodiment, this disclosure also provides Polymorph A of Compound 2 L-tartrate. In a particular embodiment, Polymorph A of Compound 2 L-tartrate exhibits an X-ray powder diffraction pattern obtained using Cu Kα radiation, having two characteristic peaks expressed in degrees 2-theta (+/−0.2) at 5.4 and 8.1. In one embodiment, Polymorph A exhibits an X-ray powder diffraction pattern obtained using Cu Kα radiation, having peaks with 2-theta values substantially in accordance with FIG. 40, upper profile. In another embodiment, Polymorph A exhibits an X-ray powder diffraction pattern obtained using Cu Kα radiation, having peaks with 2-theta values substantially in accordance with Table IX below.

TABLE IX

| Peak | Position [°2Th.] |
|---|---|
| 1 | 5.4 |
| 2 | 8.1 |

In yet another embodiment, this disclosure also provides Polymorph A of Compound 2 mesylate. In a particular embodiment, Polymorph A of Compound 2 mesylate exhibits an X-ray powder diffraction pattern obtained using Cu Kα radiation, having two, three, or four characteristic peaks expressed in degrees 2-theta (+/−0.2) selected from the group consisting of 4.0, 11.4, 11.8, and 19.8. In one embodiment, Polymorph A exhibits an X-ray powder diffraction pattern obtained using Cu Kα radiation, having peaks with 2-theta values substantially in accordance with FIG. 42. In another embodiment, Polymorph A exhibits an X-ray powder diffraction pattern obtained using Cu Kα radiation, having peaks with 2-theta values substantially in accordance with Table X below.

TABLE X

| Peak | Position [°2Th.] |
|---|---|
| 1. | 4.0 |
| 2. | 11.4 |
| 3. | 11.8 |
| 4. | 19.8 |
| 5. | 27.9 |
| 6. | 36.0 |

In yet another aspect, this disclosure is directed to a salt or cocrystal of heptadecan-9-yl 8-((2-hydroxyethyl)(8-(nonyloxy)-8-oxooctyl)amino)octanoate (“Compound 3”) and trimesic acid.

In one embodiment, this disclosure also provides Polymorph A of Compound 3 trimesate. In a particular embodiment, Polymorph A of Compound 3 trimesate exhibits an X-ray powder diffraction pattern obtained using Cu Kα radiation, having two, three, four or more characteristic peaks expressed in degrees 2-theta (+/−0.4) selected from the group consisting of 3.5, 6.8, 10.4, 18.9 and 20.9. In one embodiment, Polymorph A exhibits an X-ray powder diffraction pattern obtained using Cu Kα radiation, having peaks with 2-theta values substantially in accordance with FIG. 46. In another embodiment, Polymorph A exhibits an X-ray powder diffraction pattern obtained using Cu Kα radiation, having peaks with 2-theta values substantially in accordance with Table XI below.

TABLE XI

| Peak | Position [°2Th.] |
|---|---|
| 1. | 3.5 |
| 2. | 6.8 |
| 3. | 10.4 |
| 4. | 18.9 |
| 5. | 20.9 |
| 6. | 24.3 |
| 7. | 27.5 |

In one embodiment, this disclosure also provides Polymorph B of Compound 3 trimesate. In a particular embodiment, Polymorph B of Compound 3 trimesate exhibits an X-ray powder diffraction pattern obtained using Cu Kα radiation, comprising two, three, or more characteristic peaks expressed in degrees 2-theta (+/−0.2) selected from the group consisting of 6.2, 10.8, 16.5, and 26.7. In another embodiment, Polymorph B of Compound 3 trimesate exhibits an X-ray powder diffraction pattern obtained using Cu Kα radiation, having characteristic peaks expressed in degrees 2-theta (+/−0.2) at 6.2, 10.8, 16.5, and 26.7.

In a further embodiment, Polymorph B of Compound 3 trimesate exhibits an X-ray powder diffraction pattern obtained using Cu Kα radiation, having at least five characteristic peaks expressed in degrees 2-theta (+/−0.2), selected from the group consisting of 6.2, 10.8, 12.4, 16.5, 18.7, 22.5, and 26.7. In one embodiment, Polymorph B of Compound 3 trimesate exhibits an X-ray powder diffraction obtained using Cu Kα radiation, pattern having at least six characteristic peaks expressed in degrees 2-theta (+/−0.2), selected from the group consisting of 6.2, 10.8, 12.4, 16.5, 18.7, 22.5, and 26.7.

In a particular embodiment, Polymorph B of Compound 3 trimesate exhibits an X-ray powder diffraction pattern obtained using Cu Kα radiation, having two, three, four, or more characteristic peaks expressed in degrees 2-theta (+/−0.4) selected from the group consisting of 6.2, 10.8, 12.4, 16.5, 18.7, 22.5 and 26.7. In one embodiment, Polymorph B exhibits an X-ray powder diffraction pattern obtained using Cu Kα radiation, having peaks with 2-theta values substantially in accordance with FIG. 48. In another embodiment, Polymorph B exhibits an X-ray powder diffraction obtained using Cu Kα radiation, pattern having peaks with 2-theta values substantially in accordance with Table XII below.

TABLE XII

| Peak | Position [°2Th.] |
|---|---|
| 1. | 6.2 |
| 2. | 10.8 |
| 3. | 12.4 |
| 4. | 16.5 |
| 5. | 18.7 |
| 6. | 22.5 |
| 7. | 26.7 |

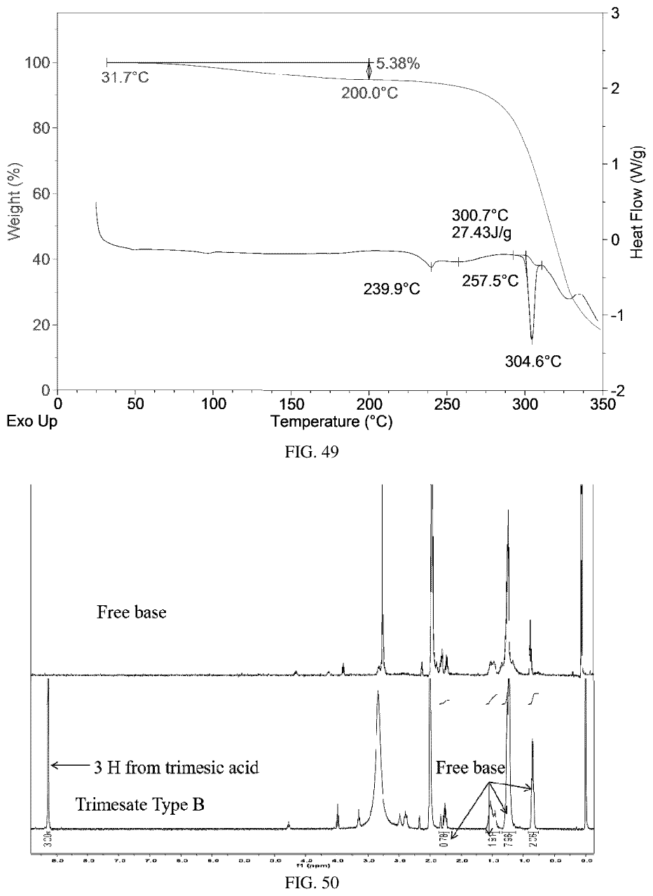

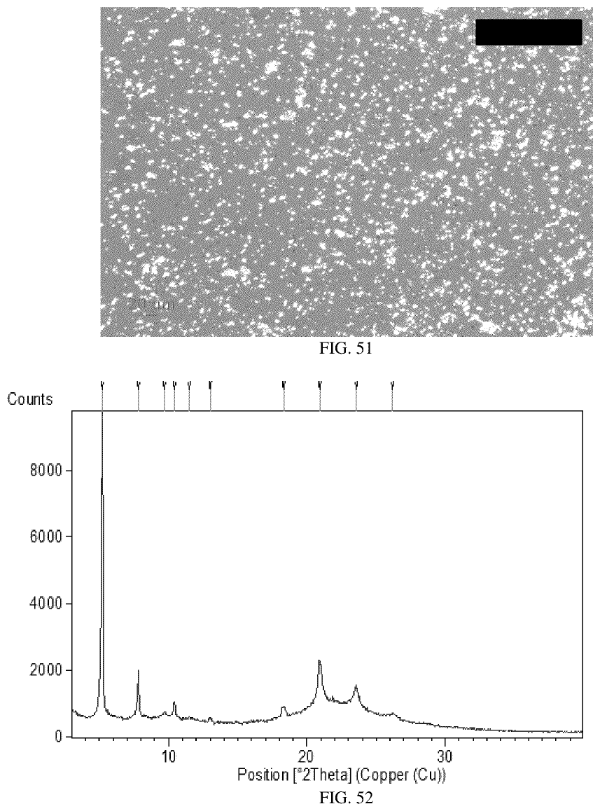

In other embodiments, Polymorph B of trimesate of Compound 3 is identifiable on the basis of a characteristic peak observed in a differential scanning calorimetry thermogram. In one embodiment, Polymorph B of trimesate of Compound 3 exhibits a differential scanning calorimetry thermogram showing a characteristic melting endotherm peak expressed in units of ° C. with an onset temperature of about 305+/−2° C. In another embodiment, Polymorph A of trimesate of Compound 3 exhibits a differential scanning calorimetry thermogram showing a second primary endotherm expressed in units of ° C. at a temperature of 240+/−2° C. In another embodiment, Polymorph B of trimesate of Compound 3 exhibits a differential scanning calorimetry thermogram substantially in accordance with FIG. 49.

In one embodiment, Polymorph A of trimesate of Compound 3 is substantially free of impurities, meaning there is not a significant amount of impurities present in the sample of Polymorph A. In another embodiment, Polymorph A is a crystalline solid substantially free of amorphous Compound 3 (or any of its amorphous salt forms). In yet another embodiment, Polymorph A is a crystalline solid substantially free of other polymorphs of 4-hydroxybenzoate of Compound 3 and substantially free of amorphous Compound 3 (or any of its amorphous salt forms). For example, Polymorph A is a crystalline solid substantially free of Polymorph B of trimesate of Compound 3 and substantially free of amorphous Compound 3 (or any of its amorphous salt forms). The skilled artisan understands that a solid sample of Polymorph B may also include other polymorphs (e.g., Polymorph A), and/or amorphous Compound 3 (or any of its amorphous salt forms).

In another embodiment, a sample of a salt or cocrystal of Compound 3 comprises Polymorph A of trimesate of Compound 3 substantially free of other polymorphs (e.g., Polymorph B of trimesate of Compound 3). As used herein, the term “substantially free of other polymorphs” means that a sample of crystalline Compound 3 trimesate contains no significant amount of other polymorphs (e.g., Polymorph B). In certain embodiments, at least about 90% by weight of a sample is Polymorph A, with only 10% being other polymorphs (e.g., Polymorph B) and/or amorphous Compound 3 (or any of its amorphous salt forms). In certain embodiments, at least about 95% by weight of a sample is Polymorph A, with only 5% being other polymorphs (e.g., Polymorph B) and/or amorphous Compound 3 (or any of its amorphous salt forms). In still other embodiments, at least about 98% by weight of a sample is Polymorph A, with only 2% by weight being other polymorphs (e.g., Polymorph B) and/or amorphous Compound 3 (or any of its amorphous salt forms). In still other embodiments, at least about 99% by weight of a sample is Polymorph A, with only 1% by weight being other polymorphs (e.g., Polymorph B) and/or amorphous Compound 3 (or any of its amorphous salt forms). In still other embodiments, at least about 99.5% by weight of a sample is Polymorph A, with only 0.5% by weight being other polymorphs (e.g., Polymorph B) and/or amorphous Compound 3 (or any of its amorphous salt forms). In still other embodiments, at least about 99.9% by weight of a sample is Polymorph A, with only 0.1% by weight being other polymorphs (e.g., Polymorph B) and/or amorphous Compound 3 (or any of its amorphous salt forms).

In certain embodiments, a sample of a salt or cocrystal of Compound 3 (e.g., Compound 3 trimesate) may contain impurities. Non-limiting examples of impurities include other polymorph forms, or residual organic and inorganic molecules such as related impurities (e.g., intermediates used to make Compound 3 or by-products), solvents, water or salts. In one embodiment, a sample of a salt or cocrystal of Compound 3, e.g., trimesate Polymorph A is substantially free from impurities, meaning that no significant amount of impurities are present. In another embodiment, a sample of the salt or cocrystal of Compound 3 contains less than 10% weight by weight (wt/wt) total impurities. In another embodiment, a sample of the salt or cocrystal of Compound 3 contains less than 5% wt/wt total impurities. In another embodiment, a sample of the salt or cocrystal of Compound 3 contains less than 2% wt/wt total impurities. In another embodiment, a sample of the salt or cocrystal of Compound 3 contains less than 1% wt/wt total impurities. In yet another embodiment, a sample of the salt or cocrystal of Compound 3 contains less than 0.1% wt/wt total impurities. In yet another embodiment, a sample of the salt or cocrystal of Compound 3 does not contain a detectable amount of impurities.

In one embodiment, this disclosure also provides Polymorph A of MC3 trimesate. In one embodiment, Polymorph A of MC3 trimesate exhibits an X-ray powder diffraction pattern obtained using Cu Kα radiation, comprising two, three, or more characteristic peaks expressed in degrees 2-theta (+/−0.2) selected from the group consisting of 5.2, 7.8, 20.9, and 23.6. In another embodiment, Polymorph A of MC3 trimesate exhibits an X-ray powder diffraction pattern obtained using Cu Kα radiation, having two, three, four, or more characteristic peaks expressed in degrees 2-theta (+/−0.2) selected from the group consisting of 5.2, 7.8, 10.4, 20.9, and 23.6. In a further embodiment, Polymorph A of MC3 trimesate exhibits an X-ray powder diffraction pattern obtained using Cu Kα radiation, having characteristic peaks expressed in degrees 2-theta (+/−0.2) at 5.2, 7.8, 10.4, 18.3, 20.9, 23.6, and 26.2.

In one embodiment, Polymorph A of MC3 trimesate exhibits an X-ray powder diffraction pattern obtained using Cu Kα radiation, having at least seven characteristic peaks expressed in degrees 2-theta (+/−0.2), selected from the group consisting of 5.2, 7.8, 9.7, 10.4, 18.3, 20.9, 23.6, and 26.2. In another embodiment, Polymorph A of MC3 trimesate exhibits an X-ray powder diffraction pattern obtained using Cu Kα radiation, having at least nine characteristic peaks expressed in degrees 2-theta (+/−0.2), selected from the group consisting of 5.2, 7.8, 9.7, 10.4, 11.5, 13.0, 18.3, 20.9, 23.6, and 26.2.

In a particular embodiment, Polymorph A of MC3 trimesate exhibits an X-ray powder diffraction pattern obtained using Cu Kα radiation, having two, three, four or more characteristic peaks expressed in degrees 2-theta (+/−0.2) selected from the group consisting of 5.2, 7.8, 10.4, 18.3, 20.9, 23.6, and 26.2. In one embodiment, Polymorph A exhibits an X-ray powder diffraction pattern obtained using Cu Kα radiation, having peaks with 2-theta values substantially in accordance with FIG. 52. In another embodiment, Polymorph A exhibits an X-ray powder diffraction pattern obtained using Cu Kα radiation, having peaks with 2-theta values substantially in accordance with Table XIII below.

TABLE XIII

| Peak | Position [°2Th.] |
|---|---|
| 1. | 5.2 |
| 2. | 7.8 |
| 3. | 9.7 |
| 4. | 10.4 |
| 5. | 11.5 |
| 6. | 13.0 |
| 7. | 18.3 |
| 8. | 20.9 |
| 9. | 23.6 |
| 10. | 26.2 |

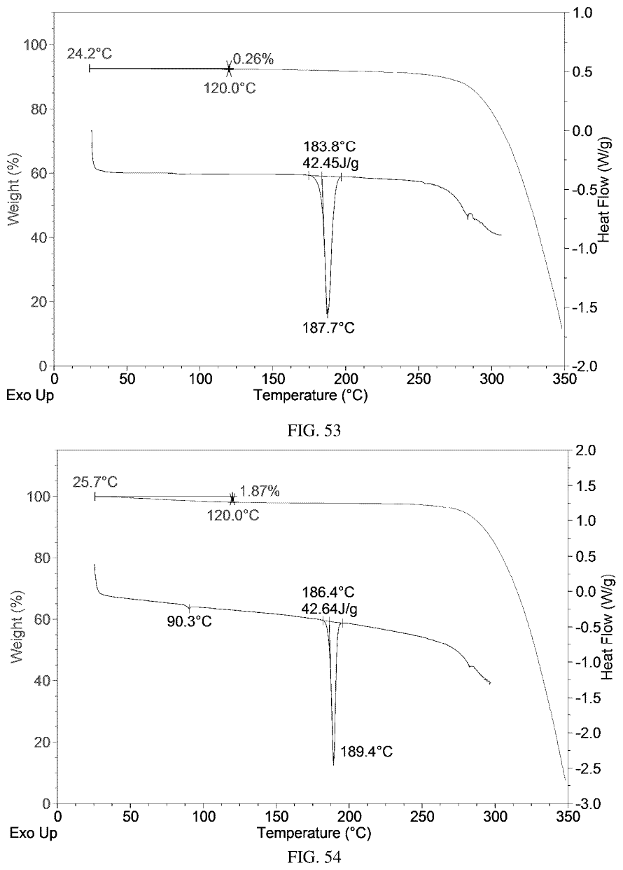

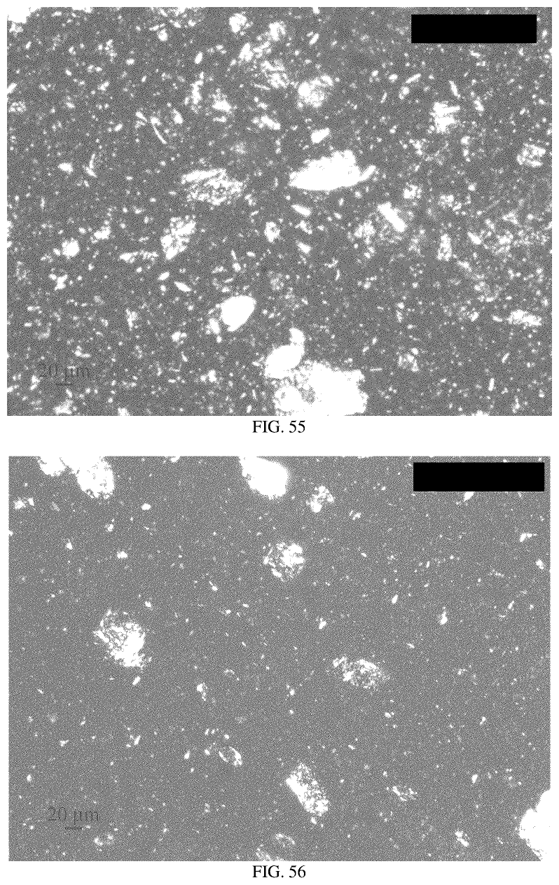

In other embodiments, Polymorph A of trimesate of MC3 is identifiable on the basis of a characteristic peak observed in a differential scanning calorimetry thermogram. In one embodiment, Polymorph A of trimesate of MC3 exhibits a differential scanning calorimetry thermogram showing a characteristic melting endotherm peak expressed in units of ° C. with an onset temperature of about 184+/−2° C. In another embodiment, Polymorph A of trimesate of MC3 exhibits a differential scanning calorimetry thermogram substantially in accordance with the lower curve shown in FIG. 53. In another embodiment, Polymorph A of trimesate of MC3 exhibits a differential scanning calorimetry thermogram showing a characteristic melting endotherm peak expressed in units of ° C. with an onset temperature of about 186+/−2° C. In another embodiment, Polymorph A of trimesate of MC3 exhibits a differential scanning calorimetry thermogram showing a second primary endotherm expressed in units of ° C. at a temperature of 90+/−2° C. In another embodiment, Polymorph A of trimesate of MC3 exhibits a differential scanning calorimetry thermogram substantially in accordance with FIG. 54.

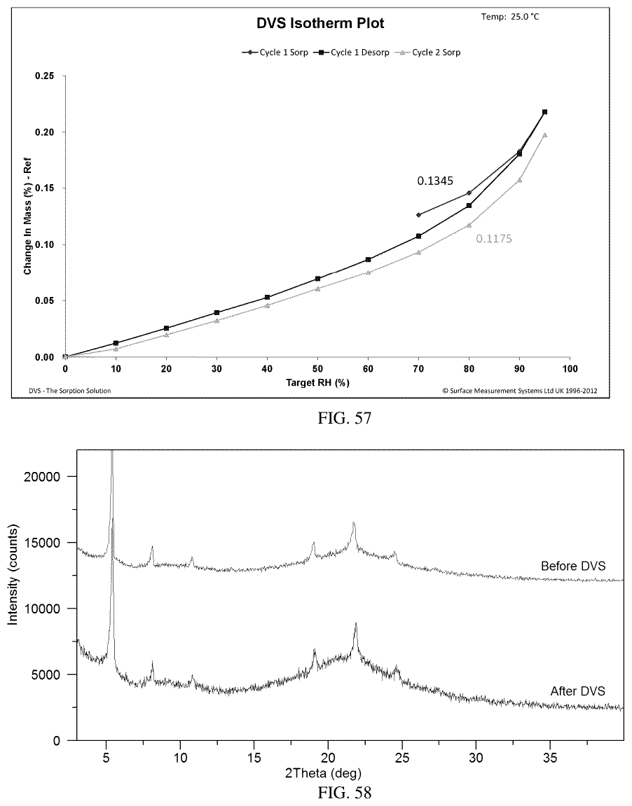

In another embodiment, provided herein is Polymorph A of trimesate of MC3, wherein the solid form undergoes a weight increase of less than 1.0% (e.g., less than 0.5%, or less than 0.3%) upon increasing relative humidity from 5.0% to 95.0% at e.g., 25° C. In another embodiment, Polymorph A of trimesate of MC3 is characterized as having a dynamic vapor sorption profile that is substantially in accordance with FIG. 57.

In one embodiment, Polymorph A of trimesate of MC3 is substantially free of impurities, meaning there is not a significant amount of impurities present in the sample of Polymorph A. In another embodiment, Polymorph A is a crystalline solid substantially free of amorphous MC3 (or any of its amorphous salt forms). In yet another embodiment, Polymorph A is a crystalline solid substantially free of other polymorphs of trimesate of MC3 and substantially free of amorphous MC3 (or any of its amorphous salt forms). For example, Polymorph A is a crystalline solid substantially free of Polymorph B of trimesate of MC3 and substantially free of amorphous MC3 (or any of its amorphous salt forms). The skilled artisan understands that a solid sample of Polymorph A may also include other polymorphs (e.g., Polymorph B), and/or amorphous MC3 (or any of its amorphous salt forms).

As used herein, the term “substantially free of amorphous MC3” means that the compound contains no significant amount of amorphous MC3 (or any of its amorphous salt forms). In another embodiment, a sample of a salt or cocrystal of MC3 comprises Polymorph A of trimesate of MC3 substantially free of other polymorphs (e.g., Polymorph B of trimesate of MC3). As used herein, the term “substantially free of other polymorphs” means that a sample of crystalline MC3 trimesate contains no significant amount of other polymorphs (e.g., Polymorph B). In certain embodiments, at least about 90% by weight of a sample is Polymorph A, with only 10% being other polymorphs (e.g., Polymorph B) and/or amorphous MC3 (or any of its amorphous salt forms). In certain embodiments, at least about 95% by weight of a sample is Polymorph A, with only 5% being other polymorphs (e.g., Polymorph B) and/or amorphous MC3 (or any of its amorphous salt forms). In still other embodiments, at least about 98% by weight of a sample is Polymorph A, with only 2% by weight being other polymorphs (e.g., Polymorph B) and/or amorphous MC3 (or any of its amorphous salt forms). In still other embodiments, at least about 99% by weight of a sample is Polymorph A, with only 1% by weight being other polymorphs (e.g., Polymorph B) and/or amorphous MC3 (or any of its amorphous salt forms). In still other embodiments, at least about 99.5% by weight of a sample is Polymorph A, with only 0.5% by weight being other polymorphs (e.g., Polymorph B) and/or amorphous MC3 (or any of its amorphous salt forms). In still other embodiments, at least about 99.9% by weight of a sample is Polymorph A, with only 0.1% by weight being other polymorphs (e.g., Polymorph B) and/or amorphous MC3 (or any of its amorphous salt forms).

In certain embodiments, a sample of a salt or cocrystal of MC3 (e.g., MC3 trimesate) may contain impurities. Non-limiting examples of impurities include other polymorph forms, or residual organic and inorganic molecules such as related impurities (e.g., intermediates used to make MC3 or by-products), solvents, water or salts. In one embodiment, a sample of a salt or cocrystal of MC3, e.g., trimesate Polymorph A is substantially free from impurities, meaning that no significant amount of impurities are present. In another embodiment, a sample of the salt or cocrystal of MC3 contains less than 10% weight by weight (wt/wt) total impurities. In another embodiment, a sample of the salt or cocrystal of MC3 contains less than 5% wt/wt total impurities. In another embodiment, a sample of the salt or cocrystal of MC3 contains less than 2% wt/wt total impurities. In another embodiment, a sample of the salt or cocrystal of MC3 contains less than 1% wt/wt total impurities. In yet another embodiment, a sample of the salt or cocrystal of MC3 contains less than 0.1% wt/wt total impurities. In yet another embodiment, a sample of the salt or cocrystal of MC3 does not contain a detectable amount of impurities.

In one embodiment, this disclosure also provides Polymorph B of MC3 trimesate. In one embodiment, Polymorph B of MC3 trimesate exhibits an X-ray powder diffraction pattern obtained using Cu Kα radiation, having two, three, four or more characteristic peaks expressed in degrees 2-theta (+/−0.2) selected from the group consisting of (+/−0.2) at 4.8, 19.4, 24.3, and 26.8. In a further embodiment, Polymorph B of MC3 trimesate exhibits an X-ray powder diffraction pattern obtained using Cu Kα radiation, having characteristic peaks expressed in degrees 2-theta (+/−0.2) at 4.8, 5.4, 7.2, 9.7, 19.4, 24.3, 26.8, and 29.3.

In one embodiment, Polymorph B of MC3 trimesate exhibits an X-ray powder diffraction pattern obtained using Cu Kα radiation, having at least seven characteristic peaks expressed in degrees 2-theta (+/−0.2), selected from the group consisting of 4.8, 5.4, 7.2, 9.7, 12.1, 19.4, 21.9, 24.3, 26.8, 29.3, and 31.8. In another embodiment, Polymorph B of MC3 trimesate exhibits an X-ray powder diffraction pattern obtained using Cu Kα radiation, having at least nine characteristic peaks expressed in degrees 2-theta (+/−0.2), selected from the group consisting 4.8, 5.4, 7.2, 9.7, 12.1, 14.5, 17.0, 19.4, 21.9, 24.3, 26.8, and 29.3.

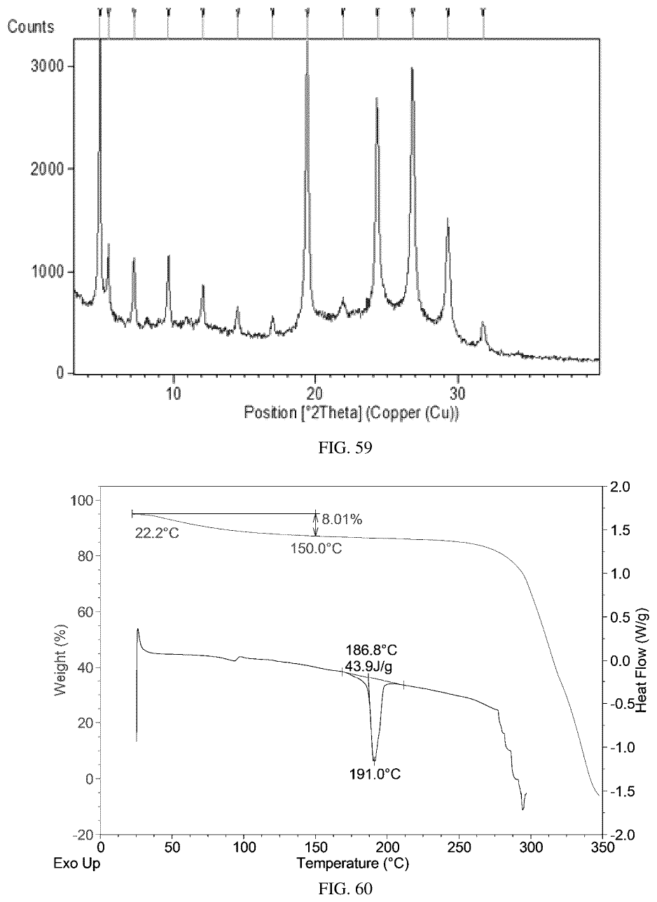

In a particular embodiment, Polymorph B of MC3 trimesate exhibits an X-ray powder diffraction pattern obtained using Cu Kα radiation, having two, three, four or more characteristic peaks expressed in degrees 2-theta (+/−0.2) selected from the group consisting of 4.8, 5.4, 7.2, 9.7, 19.4, 24.3, 26.8, and 29.3. In one embodiment, Polymorph B exhibits an X-ray powder diffraction pattern obtained using Cu Kα radiation, having peaks with 2-theta values substantially in accordance with FIG. 59. In another embodiment, Polymorph B exhibits an X-ray powder diffraction pattern obtained using Cu Kα radiation, having peaks with 2-theta values substantially in accordance with Table XIV below.

TABLE XIV

| Peak | Position [°2Th.] |
|---|---|
| 1. | 4.8 |
| 2. | 5.4 |
| 3. | 7.2 |
| 4. | 9.7 |
| 5. | 12.1 |
| 6. | 14.5 |
| 7. | 17.0 |
| 8. | 19.4 |
| 9. | 21.9 |
| 10. | 24.3 |
| 11. | 26.8 |
| 12. | 29.3 |
| 13. | 31.8 |

In other embodiments, Polymorph B of trimesate of MC3 is identifiable on the basis of a characteristic peak observed in a differential scanning calorimetry thermogram. In one embodiment, Polymorph B of trimesate of MC3 exhibits a differential scanning calorimetry thermogram showing a characteristic melting endotherm peak expressed in units of ° C. with an onset temperature of about 187+/−2° C. In another embodiment, Polymorph B of trimesate of MC3 exhibits a differential scanning calorimetry thermogram substantially in accordance with FIG. 60.

In one embodiment, Polymorph B of trimesate of MC3 is substantially free of impurities, meaning there is not a significant amount of impurities present in the sample of Polymorph B. In another embodiment, Polymorph B is a crystalline solid substantially free of amorphous MC3 (or any of its amorphous salt forms). In yet another embodiment, Polymorph B is a crystalline solid substantially free of other polymorphs of trimesate of MC3 and substantially free of amorphous trimesate of MC3 (or any of its amorphous salt forms). For example, Polymorph B is a crystalline solid substantially free of Polymorph A of trimesate of MC3 and substantially free of amorphous trimesate of MC3 (or any of its amorphous salt forms). The skilled artisan understands that a solid sample of Polymorph B may also include other polymorphs (e.g., Polymorph A), and/or amorphous MC3 (or any of its amorphous salt forms). As used herein, the term “substantially free of amorphous MC3” means that the compound contains no significant amount of amorphous MC3 (or any of its amorphous salt forms).

In another embodiment, a sample of a salt or cocrystal of MC3 comprises Polymorph B of trimesate of MC3 substantially free of other polymorphs (e.g., Polymorph A of trimesate of MC3).

As used herein, the term “substantially free of other polymorphs” means that a sample of crystalline MC3 trimesate contains no significant amount of other polymorphs (e.g., Polymorph A). In certain embodiments, at least about 90% by weight of a sample is Polymorph B, with only 10% being other polymorphs (e.g., Polymorph A) and/or amorphous MC3 (or any of its amorphous salt forms). In certain embodiments, at least about 95% by weight of a sample is Polymorph B, with only 5% being other polymorphs (e.g., Polymorph A) and/or amorphous MC3 (or any of its amorphous salt forms). In still other embodiments, at least about 98% by weight of a sample is Polymorph B, with only 2% by weight being other polymorphs (e.g., Polymorph A) and/or amorphous MC3 (or any of its amorphous salt forms). In still other embodiments, at least about 99% by weight of a sample is Polymorph B, with only 1% by weight being other polymorphs (e.g., Polymorph A) and/or amorphous MC3 (or any of its amorphous salt forms). In still other embodiments, at least about 99.5% by weight of a sample is Polymorph B, with only 0.5% by weight being other polymorphs (e.g., Polymorph A) and/or amorphous MC3 (or any of its amorphous salt forms). In still other embodiments, at least about 99.9% by weight of a sample is Polymorph B, with only 0.1% by weight being other polymorphs (e.g., Polymorph A) and/or amorphous MC3 (or any of its amorphous salt forms).

In certain embodiments, a sample of a salt or cocrystal of MC3 (e.g., MC3 trimesate) may contain impurities. Non-limiting examples of impurities include other polymorph forms, or residual organic and inorganic molecules such as related impurities (e.g., intermediates used to make MC3 or by-products), solvents, water or salts. In one embodiment, a sample of a salt or cocrystal of MC3, e.g., trimesate Polymorph B is substantially free from impurities, meaning that no significant amount of impurities are present. In another embodiment, a sample of the salt or cocrystal of MC3 contains less than 10% weight by weight (wt/wt) total impurities. In another embodiment, a sample of the salt or cocrystal of MC3 contains less than 5% wt/wt total impurities. In another embodiment, a sample of the salt or cocrystal of MC3 contains less than 2% wt/wt total impurities. In another embodiment, a sample of the salt or cocrystal of MC3 contains less than 1% wt/wt total impurities. In yet another embodiment, a sample of the salt or cocrystal of MC3 contains less than 0.1% wt/wt total impurities.

Also disclosed herein is a salt or cocrystal of an alkylated Compound 1 (structure of which is shown below, wherein R is an alkyl having, e.g., 1-20 carbon atoms) and a coformer compound such as those disclosed herein, e.g., 4-hydroxybenzoic acid, oxalic acid, trimellitic acid, orotic acid, trimesic acid, sulfuric acid, (−)-2,3-dibenzoyl-L-tartaric acid, 4-acetamido benzoic acid, (+)-L-tartaric acid, and methanesulfonic acid. For example, the salt or cocrystal of an alkylated Compound 1 has a melting point of about 50° C. or greater (e.g., about 60° C., 70° C., or greater).

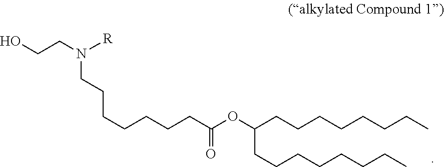

The salts or cocrystals disclosed herein may comprise Compound 1 (or Compound 2 or 3) and the coformer compound (e.g., an acid), within a ratio from 1:0.2 mol/mol to 1:5 mol/mol or from about 1:0.5 mol/mol to 1:2 mol/mol, or from 1:0.4 mol/mol to 1:1.1 mol/mol. For example, the molar ratio is about 1:1 mol/mol.

The salts or cocrystals disclosed herein may comprise (6Z,9Z,28Z,31Z)-heptatriaconta-6,9,28,31-tetraen-19-yl 4-(dimethylamino)butanoate (“MC3”) and the coformer compound (e.g., an acid), within a ratio from 1:0.5 mol/mol (i.e., 2:1 mol/mol) to 1:2 mol/mol.

The salts or cocrystals disclosed herein may be anhydrous and/or essentially solvent-free form, or be in hydrate and/or solvate form. For example, 4-hydroxybenzoate of Compound 1 is anhydrous. For example, Compound 1 orotate may be anhydrous or in a hydrate or solvate form.

Preparation of Salts or Cocrystals and Polymorphs thereof

General techniques for making polymorphs are understood by the skilled artisan. Conventionally, a salt form or cocrystal is prepared by combining in solution the free base compound and a coformer (e.g., an acid coformer) containing the anion of the salt form desired, and then isolating the solid salt or cocrystal product from the reaction solution (e.g., by crystallization, precipitation, evaporation, etc.). Other salt-forming or cocrystallization techniques may be employed.

In one aspect, provided herein is a method of preparing a salt or cocrystal of Compound 1 by combining Compound 1 with a compound selected from the group consisting of 4-hydroxybenzoic acid, oxalic acid, trimellitic acid, orotic acid, trimesic acid, and sulfuric acid. In one embodiment, the method comprises the steps: a) dissolving Compound 1 in a solvent to obtain a solution; b) combining the coformer compound with the solution; c) precipitating or crystallizing the salt or cocrystal from the solution; and d) collecting the salt or cocrystal. In one embodiment, the solvent used in step a) is n-heptane, ethyl acetate, or cyclohexane. In one embodiment, step c) is carried out substantively free of evaporation to obtain 4-hydroxybenzoate, trimellitate, orotate, and trimesate of Compound 1. In another embodiment, step c) is carried out by slow evaporation, at e.g., 5° C., to obtain, e.g., sulfate of Compound 1. In some embodiments, the molar ratio of Compound 1 and the compound is about 1:1.

Also provided herein is a method for preparing a salt or cocrystal of Compound 2 by combining Compound 2 with a compound selected from the group consisting of trimesic acid, (−)-2,3-dibenzoyl-L-tartaric acid, 4-acetamido benzoic acid, (+)-L-tartaric acid, and methanesulfonic acid. In one embodiment, the method comprises the steps: a) dissolving Compound 2 in a solvent to obtain a solution; b) combining the coformer compound with the solution; c) precipitating or crystallizing the salt or cocrystal from the solution; and d) collecting the salt or cocrystal. In one embodiment, the solvent used in step a) is n-heptane, ethyl acetate, or cyclohexane. In one embodiment, step c) is carried out substantively free of evaporation to obtain trimesate, dibenzoyl-L-tartrate, or 4-acetamido benzoate of Compound 2. In another embodiment, step c) is carried out by slow evaporation, at e.g., 5° C., to obtain, e.g., dibenzoyl-L-tartrate, L-tartrate, or mesylate of Compound 2. In some embodiments, the molar ratio of Compound 2 and the compound is about 1:1.

This disclosure also provides a method of preparing the salt or cocrystal of Compound 3 by combining Compound 3 and trimesic acid. In one embodiment, the method comprises the steps: a) dissolving Compound 3 in a solvent to obtain a solution; b) combining trimesic acid with the solution; c) precipitating or crystallizing the salt or cocrystal from the solution; and d) collecting the salt or cocrystal. In one embodiment, the solvent used in step a) is n-heptane or toluene. In one embodiment, step c) is carried out substantively free of evaporation. In another embodiment, step c) is carried out by slow evaporation. In some embodiments, the molar ratio of Compound 3 and the compound is about 1:1.

This disclosure also provides a method of preparing the salt or cocrystal of MC3 by combining MC3 and a compound selected from (+)-O,O-di-pivaloyl-D-tartaric acid (DPDT), (−)-O,O-di-pivaloyl-L-tartaric acid (DPLT), (+)-2,3-dibenzoyl-D-tartaric acid (DBDT), and trimesic acid. In one embodiment, the method comprises the steps: a) dissolving MC3 in a solvent to obtain a solution; b) combining the compound with the solution; c) precipitating or crystallizing the salt or cocrystal from the solution; and d) collecting the salt or cocrystal. In one embodiment, the solvent used in step a) is ethyl acetate, toluene, or cyclohexane. In one embodiment, step c) is carried out substantively free of evaporation. In another embodiment, step c) is carried out by slow evaporation. In some embodiments, the molar ratio of MC3 and the compound is about 1:1.

This disclosure also provides a method of preparing the salt or cocrystal of MC3 by combining MC3 and a compound selected from (+)-O,O-di-pivaloyl-D-tartaric acid (DPDT), (−)-O,O-di-pivaloyl-L-tartaric acid (DPLT), (+)-2,3-dibenzoyl-D-tartaric acid (DBDT), and trimesic acid. In one embodiment, the method comprises the steps: a) combining MC3 and trimesic acid; b) dissolving the combination of MC3 and the compound to obtain a solution; c) precipitating or crystallizing the salt or cocrystal from the solution; and d) collecting the salt or cocrystal. In one embodiment, the solvent used in step a) is ethyl acetate, toluene, or cyclohexane. In one embodiment, step c) is carried out substantively free of evaporation. In another embodiment, step c) is carried out by slow evaporation. In some embodiments, the molar ratio of MC3 and the compound is about 1:1.

In one embodiment of the method, the solvent comprises an aprotic solvent. In one embodiment of the method, the solvent comprises a nonpolar aprotic solvent. In certain embodiments, one or more of the solutions of steps a) or b) is heated. For example, the solution from step b) is subject to temperature cycling, e.g., from about 50° C. to about 5° C. (for e.g., twice, three, or four times) before step c).

Also provided herein is a process of purifying Compound 1, 2, or 3 by forming a salt or cocrystal thereof disclosed herein to separate the salt or cocrystal thereof from the impurities. The method may further comprise neutralizing the salt or cocrystal to convert to Compound 1, 2, or 3 (i.e., a free base).

Also provided herein is a process of purifying MC3 by forming a salt or cocrystal thereof disclosed herein to separate the salt or cocrystal thereof from the impurities. The method may further comprise neutralizing the salt or cocrystal to convert to MC3 (i.e., a free base).

In still another aspect, provided herein is a process of synthesizing Compound 2, Compound 3, or an analog thereof by reacting a salt or cocrystal of Compound 1 disclosed herein with a suitable electrophile, such as an ester substituted with a halogen (e.g., Br or I). The scheme below illustrates one embodiment of the process.

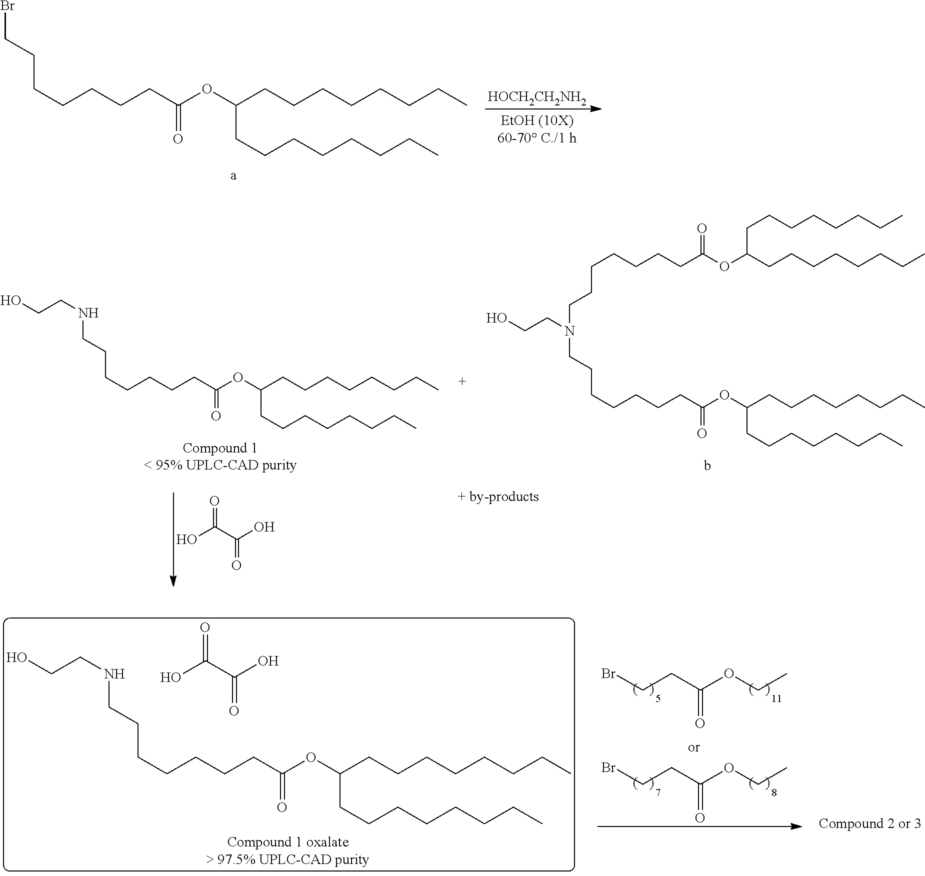

In the scheme above, Compound 1 is oil and it is hard to purify it, e.g., by separating it from a and b, and other by-products. Compound 1 oxalate is a crystal, thus is easy to separate from a, b, and/or other by-products. Forming a salt or cocrystal of Compound 1, e.g., oxalate, improves purification. Also, Compound 1 oxalate can be used to synthesize Compound 2 or 3 without converting back to Compound 1 (i.e., neutralization).

A process for synthesizing MC3 is described in Jayaraman, M.; Maximizing the Potency of siRNA Lipid Nanoparticles for Hepatic Gene Silencing In Vivo, Angew. Chem. Int. Ed. 2012, 51, 8529-8533, which is incorporated herein by reference in its entirety. MC3 corresponds to compound 16 in this article.

In one embodiment, the process of the present disclosure is advantageous as compared to other processes in that the process of the disclosure produces Compound 1, 2, or 3 or a salt or cocrystal thereof at a large scale and/or at a high purity, e.g., such that cumbersome purification (e.g., column chromatography, extraction, phase separation, distillation and solvent evaporation) is not needed. In one embodiment, the process of the present disclosure is able to process at least 100 g, 200 g, 500 g or more (e.g., 1 kg, 2 kg, 5 kg, 10 kg, 20 kg, 50 kg, 100 kg, 200 kg, 500 kg, or 1000 kg or more) Compound 1, 2, or 3 or a salt or cocrystal thereof without the need to scale up. In one embodiment, the process of the present disclosure is able to produce Compound 1, 2, or 3 or a salt or cocrystal thereof at least at a purity of at least 75%, 80%, 85%, 90%, 95%, 96%, 97%, 98%, 99%, or 99.5%, or higher. In one embodiment, the process of the present disclosure is able to produce Compound 1, 2, or 3 or a salt or cocrystal thereof with little or none impurity. In one embodiment, the impurity produced in the process of the present disclosure, even if produced, is easy to be separated from Compound 1, 2, or 3 or a salt or cocrystal thereof, without cumbersome purification (e.g., column chromatography, extraction, phase separation, distillation and solvent evaporation).

All percentages and ratios used herein, unless otherwise indicated, are by weight (i.e., weight by weight or wt/wt). Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### EXAMPLES

### X-Ray Powder Diffraction

XRPD was performed with PANalytical Empyrean, X'Pert3, and Bruker D2 X-ray powder diffractometers. The parameters used are listed in the table below.

| Parameters | XRPD | | |
|---|---|---|---|
| Model | Empyrean | X' Pert3 | Bruker D2 |
| X-Ray wavelength | Cu, kα, Kα1 (Å): 1.540598, Kα2 (Å): 1.544426 | | |
| | Kα2/Kα1 intensity ratio: 0.50 | | |
| X-Ray tube setting | 45 kV, 40 mA | | 30 kV, 10 mA |
| Divergence slit | Automatic | 1/8° | 0.6 mm |
| Scan mode | Continuous | | |
| Scan range (°2-theta) | 3-40 | | |
| Scan step time (s) | 17.8 | 46.7 | 0.1 |
| Step size (°2-theta) | 0.0167 | 0.0263 | 0.0201 |
| Scan speed (°/min) | 5 min 30 s | 5 min 04 s | 3 min 27 s |

### TGA/DSC

TGA data were collected using a TA Q500/Q5000 TGA from TA Instruments. DSC was performed using a TA Q200/Q2000 DSC from TA Instruments. Detailed parameters used are listed in the following table.

| Parameters | TGA | DSC |
|---|---|---|
| Method | Ramp | Ramp |
| Sample pan | Aluminum or platinum, | Aluminum or platinum, |
| | open | crimped |
| Temperature | RT-desired temperature; | −60° C.-desired temperature; |
| | or RT-350 ° C. | or RT-300° C. |

| | |
|---|---|
| Heating rate | 10° C./min |
| Purge gas | N₂ |

### HPLC

Agilent 1100 or Agilent 1100/1260 HPLC was utilized to analyze purity, with the detailed method listed in the table below.

| HPLC | Agilent 1100 with DAD Detector | | Agilent 1100/1260 | |
|---|---|---|---|---|
| Column | Agilent Eclipse Plus C18, | | Agilent ZORBAX SB-Phenyl, | |
| | 150 × 4.6 mm, 5 μm | | 150 × 4.6 mm, 3.5 μm | |
| Mobile phase | A: 0.1% TFA in H2O | | | |
| | B: 0.1% TFA in Acetonitrile | | | |
| Gradient table | Time (min) | % B | Time (min) | % B |
| | 0.0 | 30 | 0.0 | 10 |
| | 15.0 | 100 | 4.0 | 80 |
| | 22.0 | 100 | 6.0 | 80 |
| | 22.1 | 30 | 6.10 | 10 |
| | 25.0 | 30 | 8.0 | 10 |
| Run time | 25.0 | min | 8.0 | min |
| Post time | 0.0 | min | 0.0 | min |
| Flow rate | 0.8 | mL/min | 1.0 | mL/min |
| Injection volume | 5 | μL | 10 | μL |

| Column temperature | 40° C. | | | |
|---|---|---|---|---|
| Sampler temperature | RT | | | |
| Diluent | MeOH | | | EtOH |
| Detector | ELSD | Grace 3300 | | Detector wavelength |
| | Temperature | 50° | C. | UV at 210 nm, |
| | Flow | 2 | L/min | reference 500 nm |

| | |
|---|---|
| Gain | 1 |

Agilent 1100/1260 HPLC with Halo C18 column was utilized for purity and concentration measurements of MC3 free base, with the detailed method listed in the table below.

| Parameter | Condition | |
|---|---|---|
| Column | Halo C18, 100 × 4.6 mm, 2.7 μm | |
| Mobile phase | A: 20% NH₄HCO₃ (10 mM) + 40% MeOH + 40% THF | |
| | B: 20% IPA + 40% MeOH + 40% THF | |
| Gradient table | Time (min) | % B |
| | 0.00 | 0 |
| | 30.00 | 40 |
| | 35.00 | 50 |
| | 35.01 | 0 |
| | 40.00 | 0 |
| Run time | 40.0 | min |
| Post time | 0.0 | min |
| Flow rate | 1.0 | mL/min |
| Injection volume | 10 | μL |
| Detector wavelength | UV at 207 nm, reference 500 nm | |
| Column temperature | 40° | C. |

| | |
|---|---|
| Sampler temperature | RT |
| Diluent | EtOH |

### Dynamic Vapor Sorption

DVS was measured on via a SMS (Surface Measurement Systems) DVS Intrinsic. The relative humidity at 25° C. were calibrated against deliquescence point of LiCl, Mg(NO₃)₂ and KCl. Actual parameters for DVS test are listed in the table below.

| Parameters | DVS | |
|---|---|---|
| Temperature | 25° C. | |
| Sample size | 10~20 mg | |
| Gas and flow rate | N2, 200 mL/min | |
| dm/dt | 0.002%/min | |
| Min. dm/dt stability duration | 10 | min |
| Max. equilibrium time | 180 | min |

| | |
|---|---|
| RH range | 0% RH-95% RH |
| RH step size | 10% (0% RH-90% RH, 90% RH-0% RH) |
| | 5% (90% RH-95% RH, 95% RH-90% RH) |

¹H NMR spectrum was collected on Bruker 400M NMR Spectrometer using DMSO-d6 as solvent.

Polarized light microscopic (PLM) images were captured on Axio Lab A1 upright microscope at room temperature.

### Example 1: Salts or Cocrystals of Compound 1

### Preparation

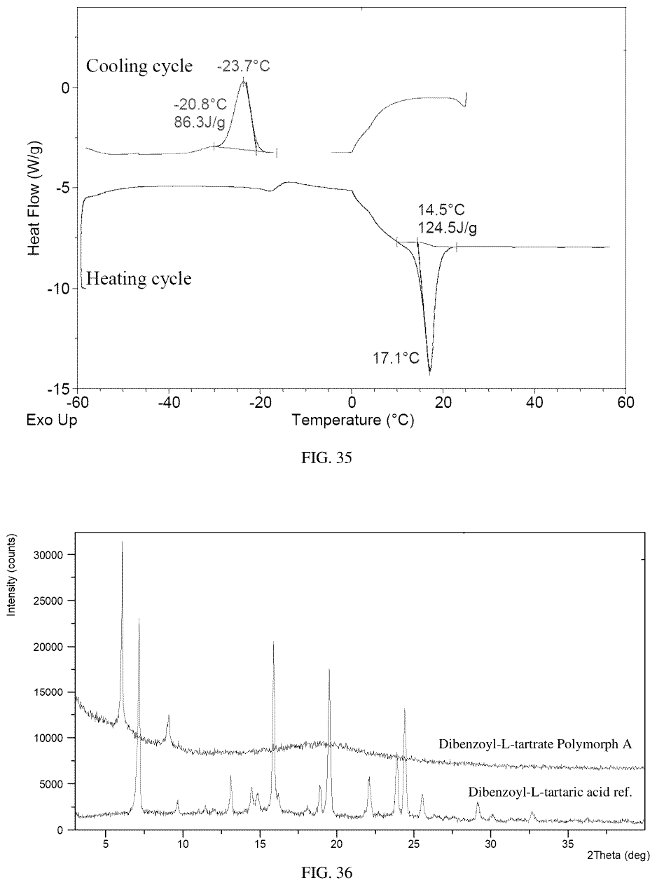

Compound 1 freebase is an oil at ambient conditions. As per the results in FIGS. 34 and 35, the freebase showed minor weight loss of 1.1% before 200° C. in TGA, and possible crystallization and melting signals in cyclic DSC, suggesting the existence of a crystalline form which melts around 17° C. (peak). Purity of the material was determined to be 99.95 area % by HPLC with ELSD detector.

To identify a crystalline salt form or cocrystal of Compound 1, screening was performed under 96 conditions using 32 acids and three solvent systems. Compound 1 freebase was dispersed in selected solvent with a 1.5-mL glass vial and corresponding salt former was added with a molar charge ratio of 1:1. The mixtures of freebase and the coformer compound (e.g., an acid) were first transferred to temperature cycling from 50° C. to 5° C. for two cycles (heating rate of 4.5° C./min, cooling rate of 0.1° C./min) and then stirred at 5° C. to induce precipitation. If the samples were still clear, they would be subjected to evaporation at different temperatures (5° C. or RT) to dryness. Resulted solids were isolated and analyzed.

Isolated crystal solids were characterized by X-ray powder diffraction (XRPD), thermo-gravimetric analysis (TGA) and differential scanning calorimetry (DSC), with proton nuclear magnetic resonance (¹H NMR) to confirm the freebase chemical structure and also potential co-existence with some organic acids. Exemplary data from the initial findings are summarized in Table 1.

TABLE 1

| | | n-Heptane | EtOAc | Cyclohexane |
|---|---|---|---|---|
| 1 | Hexanoic acid | Amorphous* | Amorphous* | Oil** |
| 2 | Fumaric acid | Acid + two extra peaks | Acid + two extra | Acid + two extra |
| | | | peaks* | peaks |
| 3 | Adipic acid | Amorphous* | Amorphous* | Acid + one extra |
| | | | | peak** |
| 4 | Suberic acid | Amorphous* | Acid* | Oil** |
| 5 | Cinnamic acid | Amorphous* | Amorphous* | Oil** |
| 6 | Benzoic acid, 4-acetamido | Acid | Two peaks* | Acid |
| 7 | (S)-Mandelic acid | Two peaks* | Two peaks* | Oil** |
| 8 | (−)-O,O-Di-pivaloyl-L-tartaric | Amorphous* | Amorphous* | Oil** |
| | acid | | | |
| 9 | Terephthalic acid | Acid | Acid | Acid |
| 10 | Trimesic acid | Amorphous | Trimesate Polymorph | Oil** |
| | | | A | |
| 11 | Citric acid | Two peaks* | Amorphous* | Two peaks' |
| 12 | Succinic acid | Two peaks* | Two peaks* | Two peaks ** |
| 13 | Malonic acid | Amorphous* | Amorphous* | Oil** |
| 14 | (+)-Camphor-10-sulfonic acid | Amorphous* | Amorphous* | Oil** |
| 15 | Nicotinic acid | Amorphous* | Acid* | Oil** |
| 16 | (+)-L-tartaric acid | Two peaks | Two peaks* | Oil** |
| 17 | p-Toluenesulfonic acid | Amorphous* | Two peaks* | Oil** |
| 18 | Hydrochloric acid | Amorphous* | Amorphous* | Amorphous |
| 19 | Sulfuric acid | Sulfate Polymorph A* | Amorphous* | Oil** |
| 20 | Phosphoric acid | Two peaks* | Amorphous* | Oil** |
| 20 | Acetic acid | Amorphous* | Amorphous* | Oil** |
| 21 | Methanesulfonic acid | Amorphous* | Amorphous* | Oil** |
| 22 | Sebacic acid | Sebacic acid | Sebacic acid* | Sebacic acid* |
| 23 | Benzoic acid | Amorphous* | Amorphous* | Amorphous* |
| 24 | 1,2,4-Trimellitic acid | Trimellitate Polymorph | Trimellitate | Trimellitate |
| | | A | Polymorph A | Polymorph A |
| 25 | Phthalic acid | Oil* | Oil* | Oil* |
| 26 | Isophthalic acid | Isophthalic acid | Isophthalic acid | Isophthalic acid |
| 27 | Orotic acid | Orotate Polymorph A | Orotate Polymorph A | Orotate Polymorph A |
| 28 | 4-Hydroxybenzoic acid | 4-Hydroxybenzoate | 4-Hydroxybenzoate | 4-Hydroxybenzoate |
| | | Polymorph A | Polymorph A | Polymorph A |
| 29 | (−)-Dibenzoyl-L-tartaric acid | Weakly crystalline | Amorphous* | Weakly crystalline |
| 30 | 2,5-Dihydroxybenzoic acid | Oil* | Oil* | 2,5- |
| | | | | Dihydroxybenzoic |
| | | | | acid |
| 31 | 2-Hydroxy benzoic acid | Oil** | Oil** | Oil** |
| 32 | 3-Hydroxy benzoic acid | Oil** | Oil** | Oil** |

*clear solutions obtained after 5° C. stirring were transferred to 5° C. evaporation.
**clear solutions obtained after 5° C. stirring were slow evaporated at RT.

Among them, five crystalline hits were discovered, including 4-hydroxybenzoate, trimellitate, orotate, trimesate and sulfate. Table 2 summarizes the properties of certain polymorphs of the salts or cocrystals.

TABLE 2

| | 4-Hydroxybenzoate | Trimellitate | Orotate | |
|---|---|---|---|---|
| | Polymorph A | Polymorph A | Polymorph A | Polymorph B |
| Appearance | White powder | Wax-like solid | Wax-like solid | |
| Solid form | Anhydrate | Hydrate | Anhydrate/Hydrate | Hydrate/solvate |
| Crystallinity | High | Medium | Medium | |
| Purity, area % | 99.96 | 99.97 | — | 99.97 |
| TGA weight loss, | 0.7-1.7 | 1.5-3.4 | 4.0 | 4.0 |
| % | | | | |
| DSC endotherm, | 66.8, 101.8 (batch 1) | 78.3, 137.1 (batch 1) | 78.8*, 85.1*, 176.3* | 83.5* |
| ° C. (onset) | 68.2, 103.5 (batch 2) | 80.0*, 137.1 (batch 2) | | |
| Hygroscopicity | Non-hygroscopic | Slightly hygroscopic | | Hygroscopic |
| (form change after | (no) | (no) | | (convert to orotate |
| DVS) | | | | Polymorph A) |

*peak temperature.
—: no data available.

Three crystalline polymorphs of Compound 1 (4-hydroxybenzoate Polymorph A, trimellitate Polymorph A and orotate Polymorph B) were prepared to larger scale for further investigation, with the detailed procedure shown below:
1. About 100 mg of freebase Compound 1 was added into a 3-mL glass vial;
2. Add corresponding acids (molar charge ratio is 1:1) into the vial;
3. Add 0.5 mL of solvent and transfer the suspension to temperature cycling from 50° C. to 5° C. (cooling rate of 0.1° C./min, two cycles) with magnetic stirring.
4. Centrifuge to isolate solids and vacuum dry at RT.

### Characterization of 4-Hydroxybenzoate

Two batches of 4-hydroxybenzoate Polymorph A (or Type A) (batch Nos. 1 and 2) were prepared by slurry in n-heptane and showed high crystallinity as characterized by XRPD in FIG. 1. The ¹H NMR of sample (batch No. 2) was collected with spectrum shown in FIG. 2. Besides freebase, a certain amount of 4-hydroxybenzic acid was detected in ¹H NMR (signals around 6.7 and 7.7 ppm), indicating the possibility of salt formation.

As indicated by the TGA and DSC data in FIG. 3, sample (batch No. 2) showed a weight loss of 0.7% up to 140° C. and two sharp endothermic peaks at 68.2° C. and 103.5° C. (onset temperature) before decomposition. Based on the negligible weight loss in TGA, 4-hydroxybenzoate Polymorph A was considered to be an anhydrous form. In addition, the two sharp endothermic signals in DSC curve implied the possible existence of another anhydrous form at higher temperature.

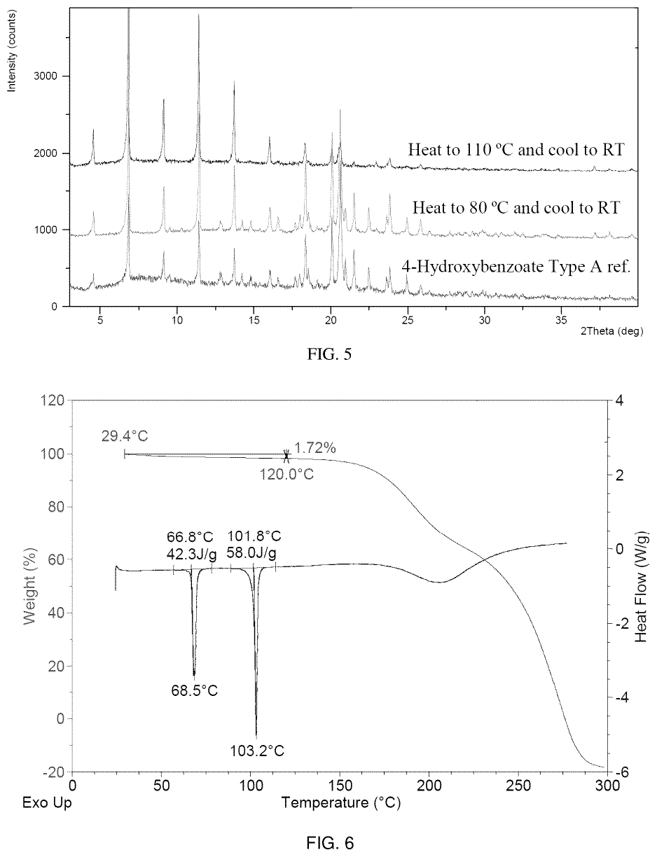

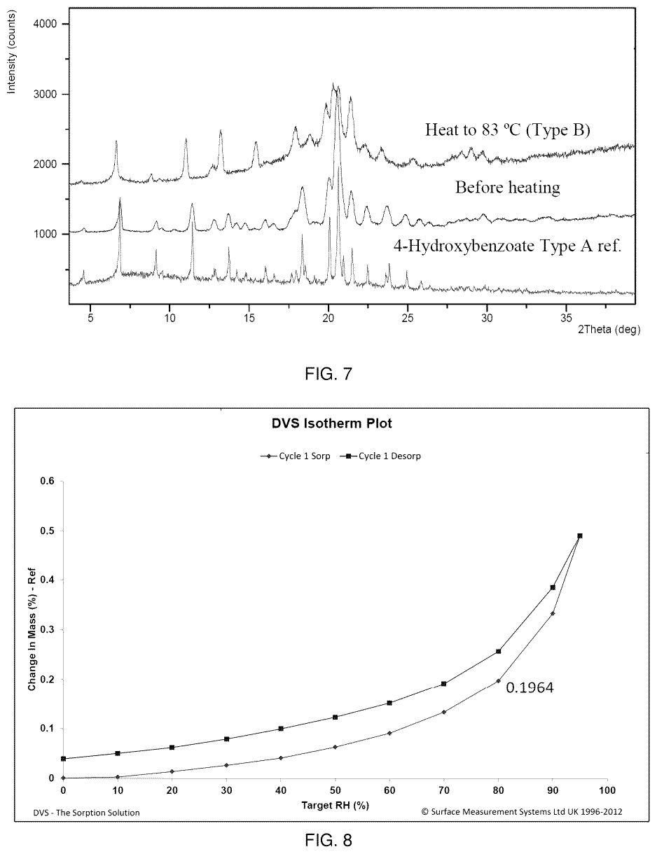

As evidenced by heating experiments in FIG. 5 and VT-XRPD results in FIGS. 6 and 7, form change (new form assigned as 4-hydroxybenzoate Polymorph B) was observed after heating sample (batch No. 1) to 83° C. (over the first endotherm in DSC) in VT-XRPD test and no form change was observed after heating sample (batch No. 2) over the first endotherm and cooling back to RT. Considering results of heating experiments and thermal signals in cyclic DSC (FIG. 4), 4-hydroxybenzoate Polymorphs A and B are possibly enantiotropically related and Polymorph A is more stable at lower temperature (RT).

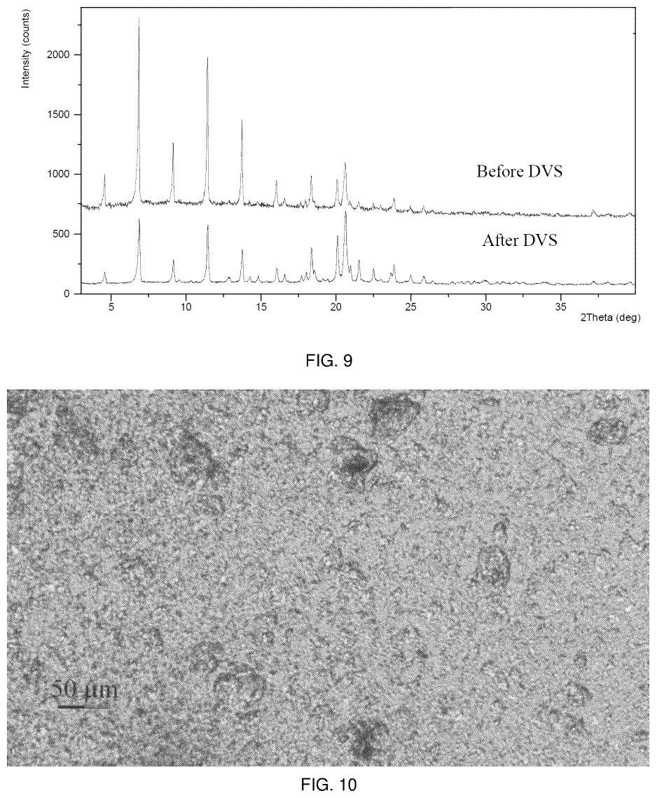

Further evaluation on hygroscopicity of 4-hydroxybenzoate Polymorph A was conducted via DVS isotherm collection at 25° C. Results in FIGS. 8 and 9 showed that sample (batch No. 1) is non-hygroscopic with no form change before and after DVS test. Moreover, sample (batch No. 1) showed aggregation of small particles (<10 m) in PLM image (FIG. 10) and a purity of 99.96 area % determined by HPLC (Table 3).

TABLE 3

| # Peak | Time (min) | RRT | Area (mAU*S) | Area (%) |
|---|---|---|---|---|
| 1 | 16.58 | 1.00 | 2070.9 | 99.96 |
| 2 | 16.99 | 1.02 | 0.8 | 0.04 |

### Characterization of Trimellitate

Trimellitate Polymorph A samples (batch Nos. 1 and 2) were prepared by reactive crystallization in EtOAc with XRPD patterns shown in FIG. 11. The ¹H NMR spectrum was collected for sample (batch No. 2) and is shown in FIG. 12. Compared to freebase, a certain amount of trimellitic acid was detected (signals between 8.0 and 9.0 ppm), indicating the salt formation.

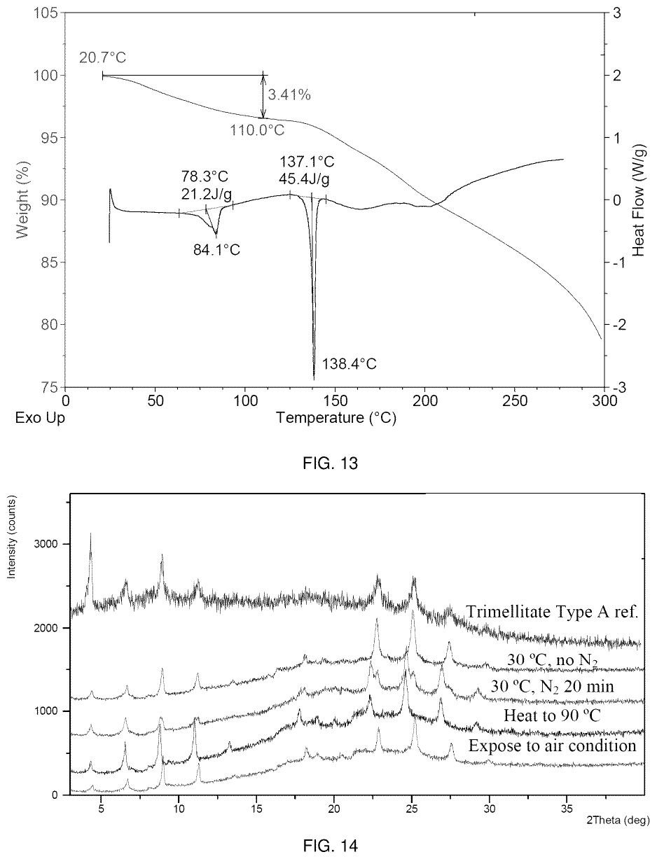

As per the TGA and DSC data in FIG. 13, sample (batch No. 1) showed a weight loss of 3.4% up to 110° C. and two endothermic peaks at 78.3° C. and 137.1° C. (onset temperature) before decomposition. As demonstrated by VT-XRPD results in FIG. 14, extra diffraction peaks appeared after 20 minutes of N₂ flow, and new form was observed at 90° C., which converted back to trimellitate Polymorph A after being heated and exposed to ambient condition, suggesting that Polymorph A is a hydrated form.

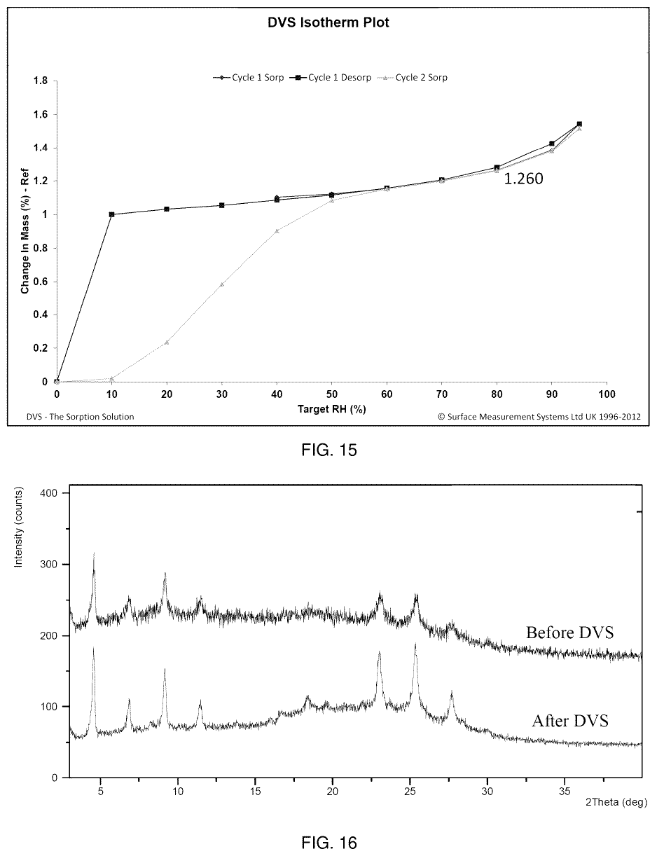

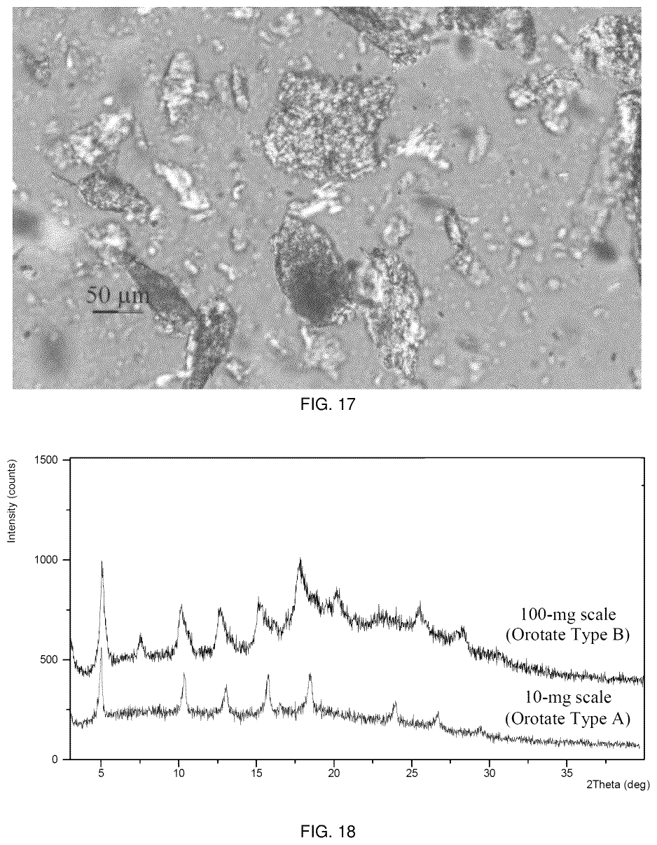

Further evaluation on hygroscopicity of trimellitate Polymorph A was performed via DVS isotherm collection at 25° C. Results in FIGS. 15 and 16 showed that sample (batch No. 1) is slightly hygroscopic with no form change before and after DVS test. Platform observed in DVS plot (FIG. 15) also indicated that Polymorph A is a hydrated form. Moreover, sample (batch No. 1) showed irregular particles (<10 μm) in PLM image (FIG. 17) and a purity of 99.97 area % determined by HPLC (Table 4).

TABLE 4

| # Peak | Time (min) | RRT | Area (mAU*S) | Area (%) |
|---|---|---|---|---|
| 1 | 16.62 | 1.00 | 1404.2 | 99.97 |
| 2 | 16.99 | 1.02 | 0.5 | 0.03 |

### Characterization of Orotate

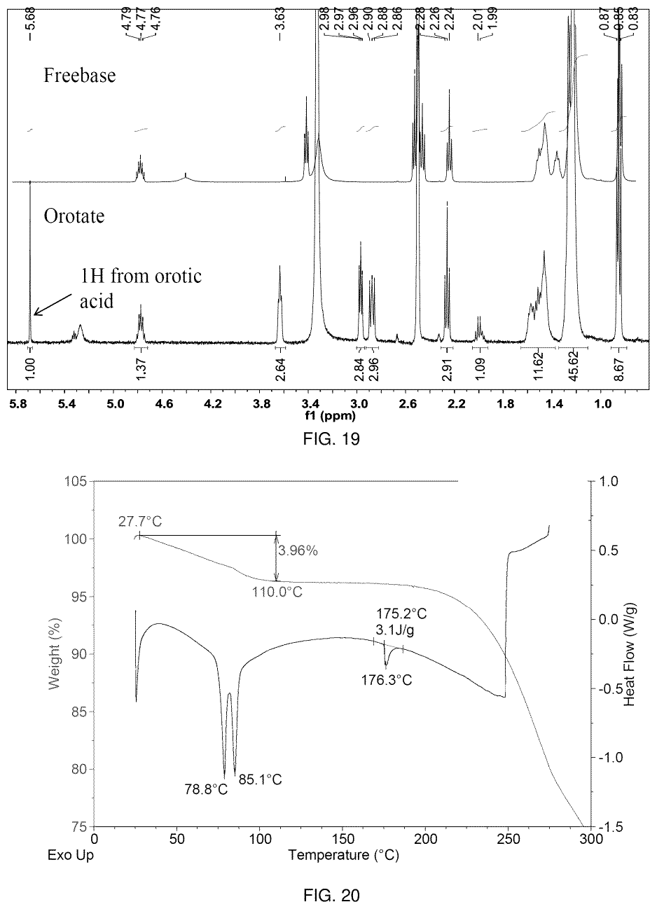

Orotate Polymorph A and Polymorph B were generated via reactive crystallization in EtOAc with XRPD patterns shown in FIG. 18. The ¹H NMR spectrum of Polymorph A was collected and is shown in FIG. 19. In addition to freebase, a certain amount of orotic acid was detected (signal at 5.7 ppm).

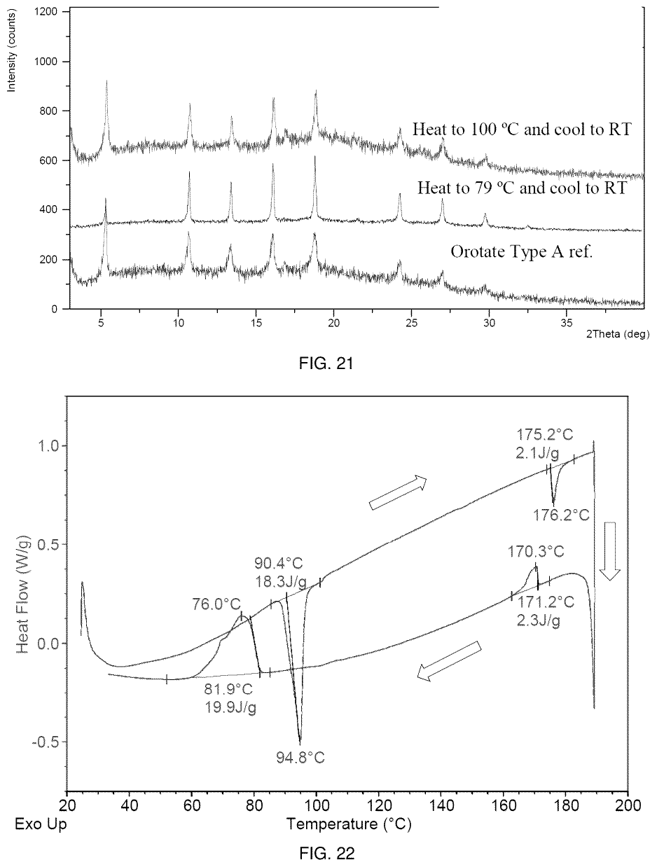

As per the TGA and DSC data in FIG. 20, Polymorph A sample showed a weight loss of 4.0% up to 110° C. and endothermic peaks at 78.8, 85.1 and 176.3° C. (peak temperature) before decomposition. Results of heating experiments in FIG. 21 showed that no form change was observed after heating Polymorph A sample over the first two endothermic signals and cooling back to RT, suggesting Polymorph A is anhydrous or a hydrated form which can rapidly absorb water at ambient conditions after de-hydration. In addition, as evidenced by the heating-cooling DSC curve of Polymorph A in FIG. 22, endothermic and exothermic signals with similar enthalpy were observed at 170~175° C. and 80~90° C., suggesting the possible form transition and the existence of anhydrate form at higher temperature.

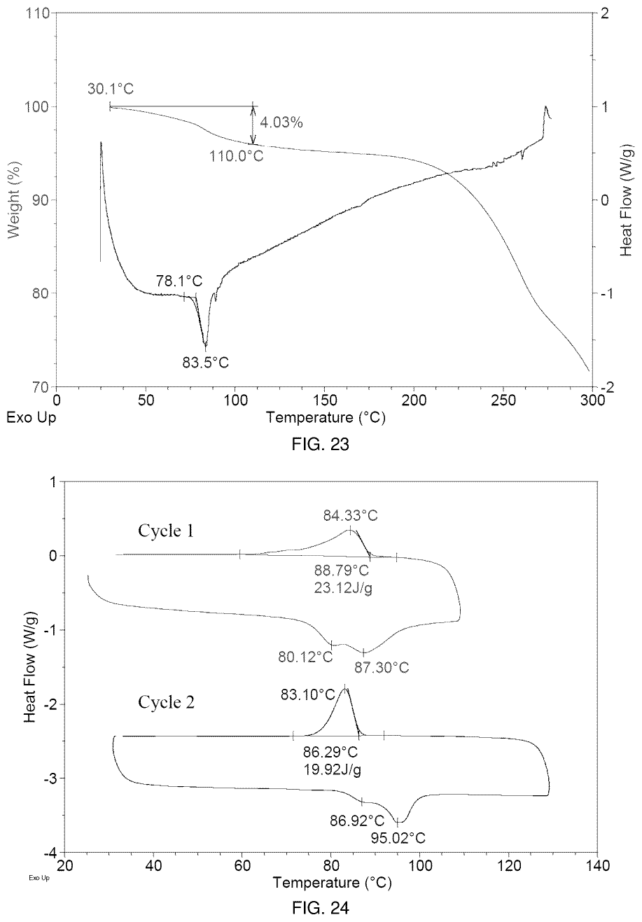

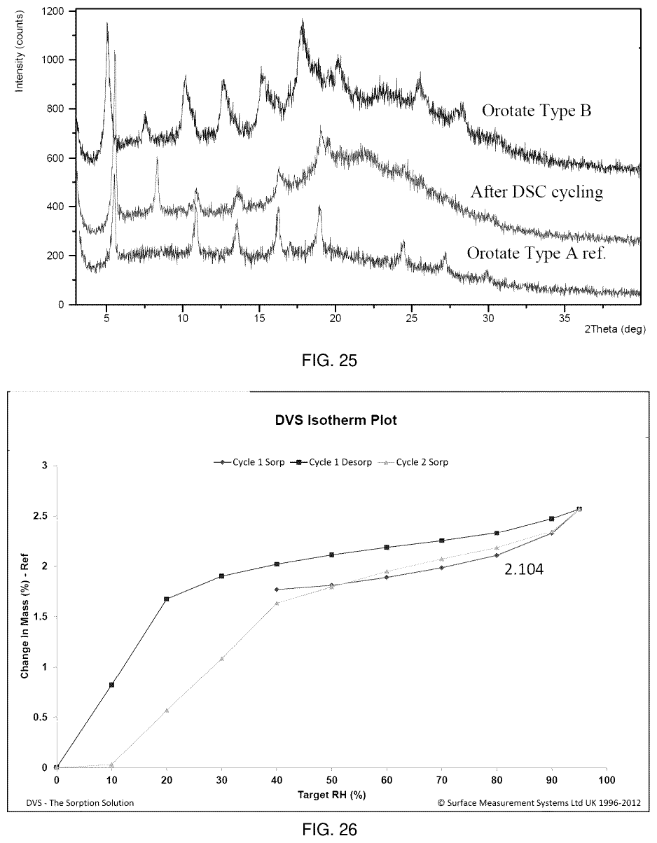

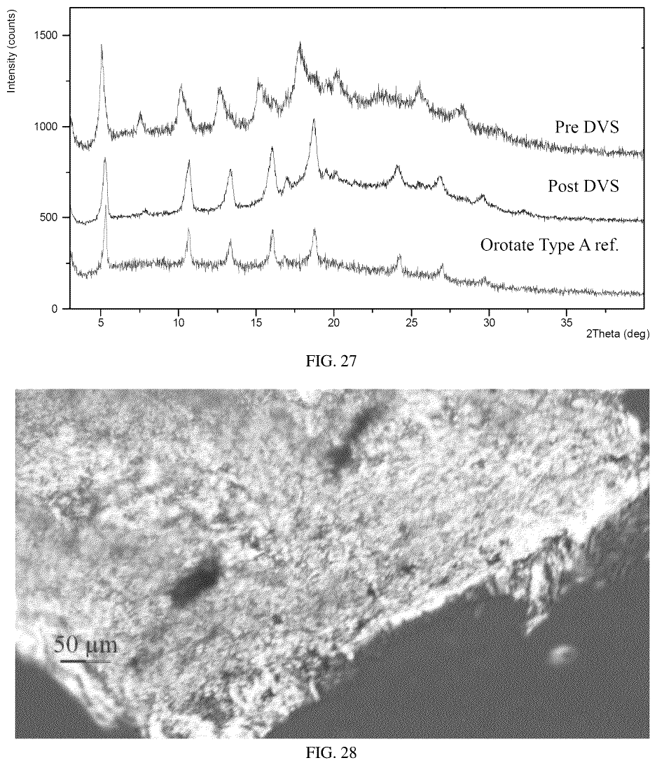

TGA and DSC data of Polymorph B in FIG. 23 showed a weight loss of 4.0% up to 110° C. and endothermic peak at 78.1° C. (onset) before decomposition. After cyclic DSC between 25° C. and 130° C., Polymorph B converted to Polymorph A with data illustrated in FIG. 24 and FIG. 25, indicating Polymorph B is a hydrated or solvate form. DVS test of Polymorph B sample showed that it is slightly hygroscopic and converted to Polymorph A after DVS test, with data displayed in FIG. 26 and FIG. 27. Also, Polymorph B sample showed irregular particles in PLM image (FIG. 28) and a purity of 99.97 area % detected by HPLC (Table 5).

TABLE 5

| # Peak | Time (min) | RRT | Area (mAU*S) | Area (%) |
|---|---|---|---|---|
| 1 | 16.62 | 1.00 | 1464.2 | 99.97 |
| 2 | 17.00 | 1.02 | 0.5 | 0.03 |

### Characterization of Sulfate

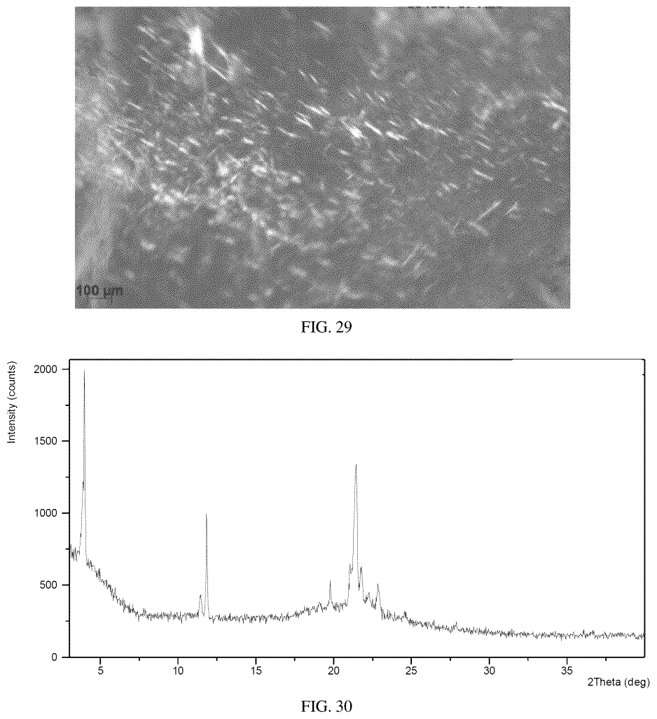

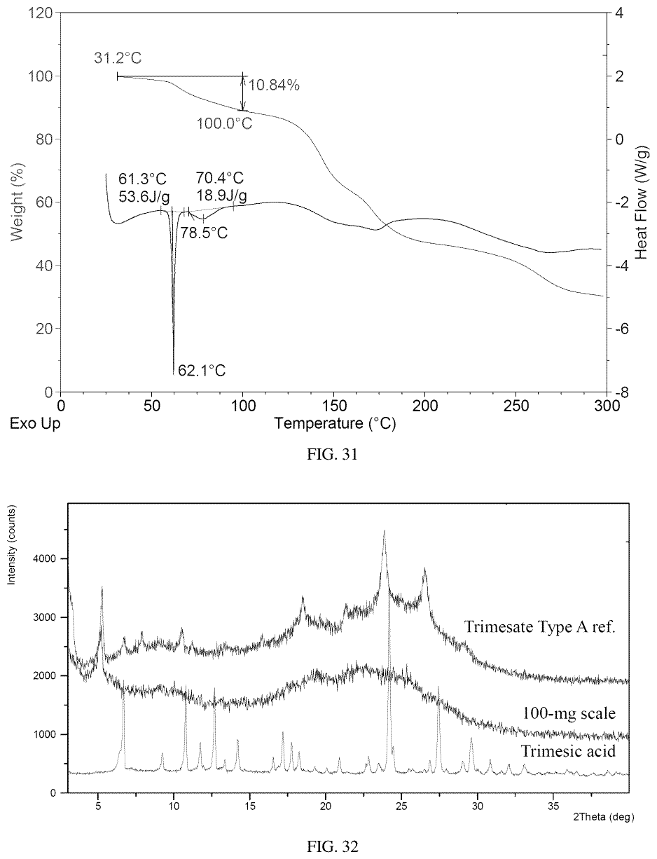

Sulfate Polymorph A was generated by slow evaporation at 5° C. in n-heptane. Needle like crystals were observed during evaporation (FIG. 29), which was further isolated for XRPD, TGA and DSC tests. Results in FIGS. 30 and 31 showed that the sample is crystalline with continuous weight loss and multiple endotherms.

### Characterization of Trimesate

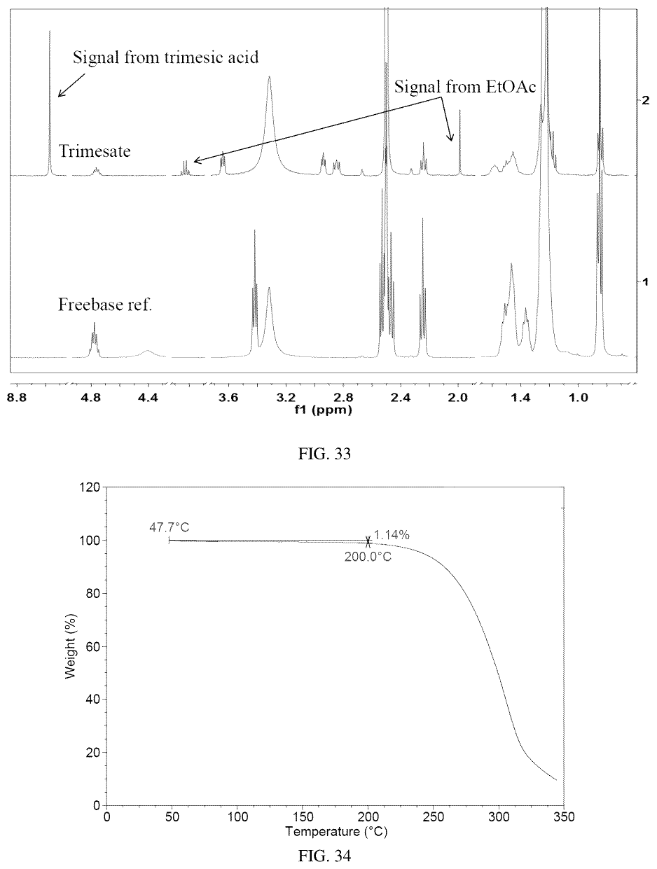

Trimesate Polymorph A was generated from reactive crystallization in EtOAc system and XRPD pattern is shown in FIG. 32. ¹H NMR results in FIG. 33 showed obvious signal of trimesic acid besides chemical shifts of freebase.

### Characterization of Oxalate

Compound 1 Oxalate was generated from recrystallization. A purity of >97.5 area % detected by UPLC-CAD.

### Example 2: Salts or Cocrystals of Compound 2

### Preparation

Compound 2 freebase showed minor weight loss of 1.6% before reaching 200° C. in TGA. No obvious glass transition signal was observed and multiple endothermic peaks were observed with temperature elevated from −60 to 35° C. Two endothermic signals at −47.7 and −34.0° C. (onset) were observed during temperature elevated from −60 to 35° C.

Similar to the process described in Example 1, to identify a crystalline salt form or cocrystal of Compound 2, screening was performed under 93 conditions using 31 acids and three solvent systems. 0.3 mL stock solutions of Compound 2 freebase (-50 mg/mL) was dispersed in selected solvent and corresponding salt former was added with a molar charge ratio of 1:1. The mixtures of freebase and the coformer compound (e.g., an acid) were first transferred to temperature cycling from 50° C. to 5° C. for three cycles (heating rate of 4.5° C./min, cooling rate of 0.1° C./min) and then stored at 5° C. before analysis. If the samples were still clear, they would be subjected to slow evaporation at 5° C. to dryness. Resulted solids were isolated and analyzed.

Isolated crystal solids were characterized by X-ray powder diffraction (XRPD), thermo-gravimetric analysis (TGA) and differential scanning calorimetry (DSC), with proton nuclear magnetic resonance (¹H NMR) to confirm the freebase chemical structure and also potential co-existence with some organic acids. Exemplary data from the initial findings are summarized in Table 6.

TABLE 6

| | | Solvent | | |
|---|---|---|---|---|
| # | Acid | n-Heptane | Cyclohexane | EtOAc |
| 1 | Trimesic acid | Trimesate Polymorph A | Trimesate Polymorph A | Gel |
| 2 | Trimellitic acid | Amorphous + acid | Amorphous | Gel |
| 3 | (−)-2,3-Dibenzoyl-L- | Dibenzoyl-L-tartrate | Dibenzoyl-L-tartrate | Dibenzoyl-L-tartrate |
| | tartaric acid | Polymorph A | Polymorph A* | Polymorph A* |
| 4 | Fumaric acid | Amorphous + two peaks | Acid | Gel |
| 5 | Terephthalic acid | Acid | Acid | Gel |
| 6 | Phthalic acid | Gel | Gel | Gel |
| 7 | Isophthalic acid | Acid | Acid | Gel |
| 8 | Benzoic acid | Gel | Gel | Gel |
| 9 | Cinnamic acid | Gel | Gel | Gel |
| 10 | 4-Hydroxy benzoic acid | Amorphous | Gel | Gel |
| 11 | Salicylic acid | Gel | Gel | Gel |
| 12 | Adipic acid | Acid | Gel | Gel |
| 13 | Suberic acid | Acid | Acid | Gel |
| 14 | Sebacic acid | Gel | Acid | Acid |
| 15 | 4-Acetamido benzoic acid | 4-Acetamido benzoate | Acid | Acid |
| | | Polymorph A + acid | | |
| 16 | S-(+)-Mandelic | Gel | Gel | Gel |
| 17 | Orotic acid | Gel | Acid | Acid |
| 18 | Hexanoic acid | Gel | Gel | Gel |
| 19 | Citric acid | Gel | Gel | Gel |
| 20 | Acetic acid | Gel | Gel | Gel |
| 21 | Succinic acid | Acid | Acid | Gel |
| 22 | Malonic acid | Gel | Gel | Gel |
| 23 | (+)-Camphor-10-sulfonic | Gel | Gel | Gel |
| | acid | | | |
| 24 | Nicotinic acid | Acid | Acid | Acid |
| 25 | (+)-L-tartaric acid | L-Tartrate Polymorph A* | Gel | L-Tartrate Polymorph |
| | | | | A* |
| 26 | Hydrochloric acid | Gel | Gel | Gel |
| 27 | Sulfuric acid | Gel | Gel | Gel |
| 28 | Phosphoric acid | Gel | Gel | Gel |
| 29 | Methanesulfonic acid | Mesylate Polymorph A* | Mesylate Polymorph A* | Gel |
| 30 | p-Toluene sulfonic acid | Gel | Gel | Gel |
| 31 | 2,5-Dihydroxybenzoic | Gel | Gel | Gel |
| | acid | | | |

*solids obtained after 5° C. evaporation.

Characterization of dibenzoyl-L-tartrate

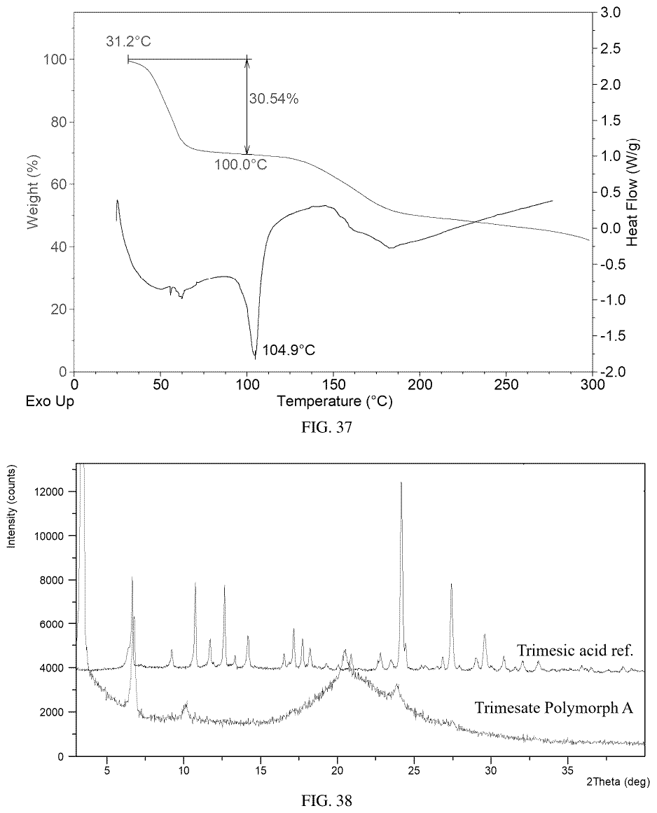

Compound 2 dibenzoyl-L-tartrate Polymorph A was prepared by combining Compound 2 freebase with (−)-2,3-dibenzoyl-L-tartaric acid in n-heptane and showed crystallinity as characterized by XRPD in FIG. 36. The TGA/DSC data as shown in FIG. 37 indicate a weight loss of 30.5% up to 100° C. and broad endothermic signals before decomposition.

### Characterization of Trimesate

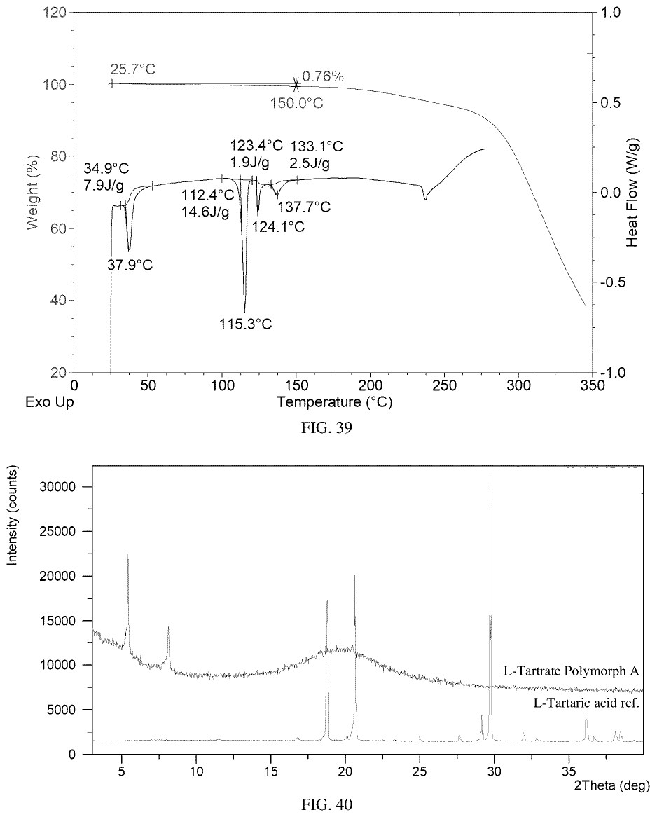

Compound 2 trimesate Polymorph A was prepared by combining Compound 2 freebase with trimesic acid in n-heptane and showed crystallinity as characterized by XRPD in FIG. 38. The TGA/DSC data as shown in FIG. 39 indicate a weight loss of 0.8% up to 150° C. and multiple endothermic signals before decomposition.

### Characterization of L-tartrate

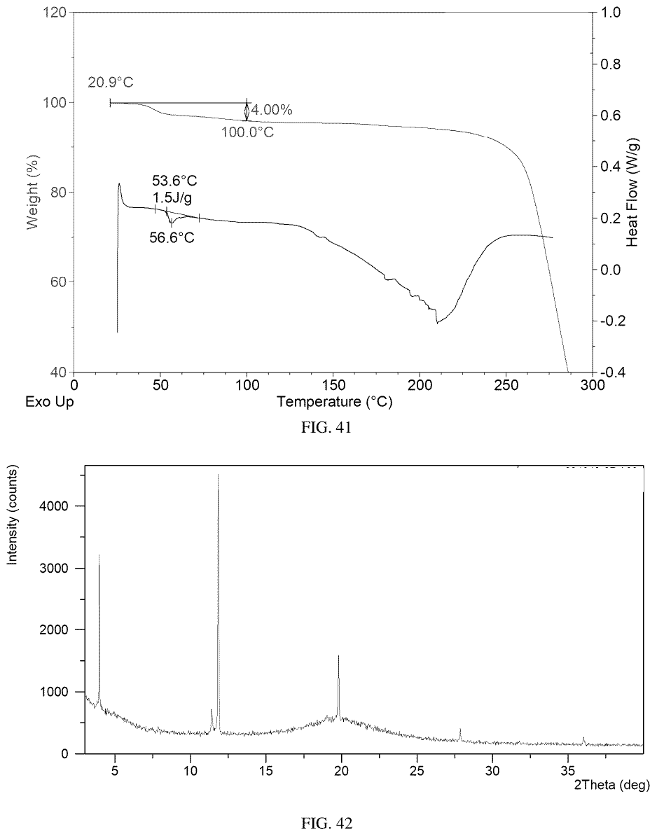

Compound 2 L-tartrate Polymorph A was prepared by combining Compound 2 freebase with L-tartaric acid in n-heptane and showed crystallinity as characterized by XRPD in FIG. 40. The TGA/DSC data as shown in FIG. 41 indicate a weight loss of 4.0% up to 100° C. and multiple endothermic signals before decomposition.

### Characterization of Mesylate

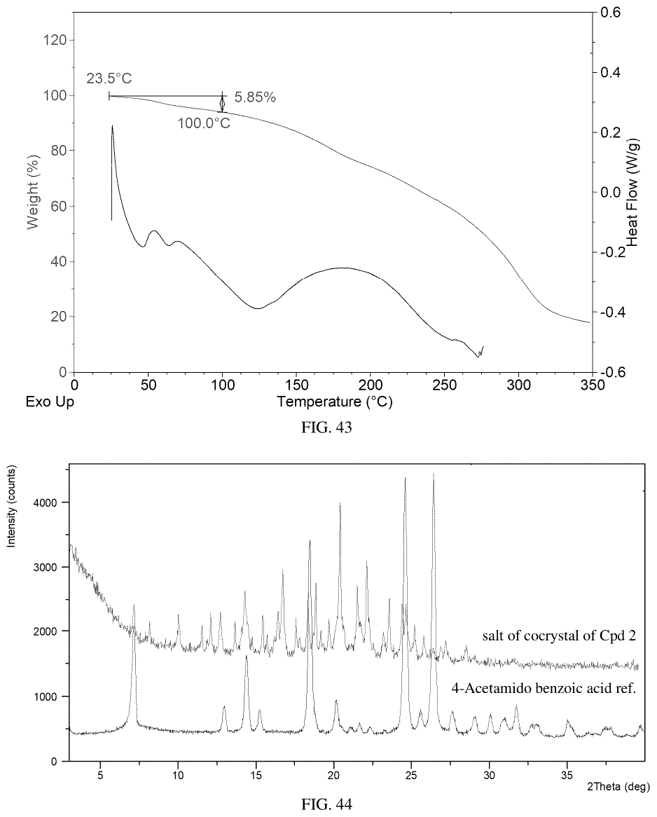

Compound 2 mesylate Polymorph A was prepared by combining Compound 2 freebase with methanesulfonic acid in n-heptane and showed crystallinity as characterized by XRPD in FIG. 42. The TGA/DSC data as shown in FIG. 43 indicate a weight loss of 5.9% up to 100° C. and irregular signals in the DSC curve.

Characterization of 4-acetamido Benzoate

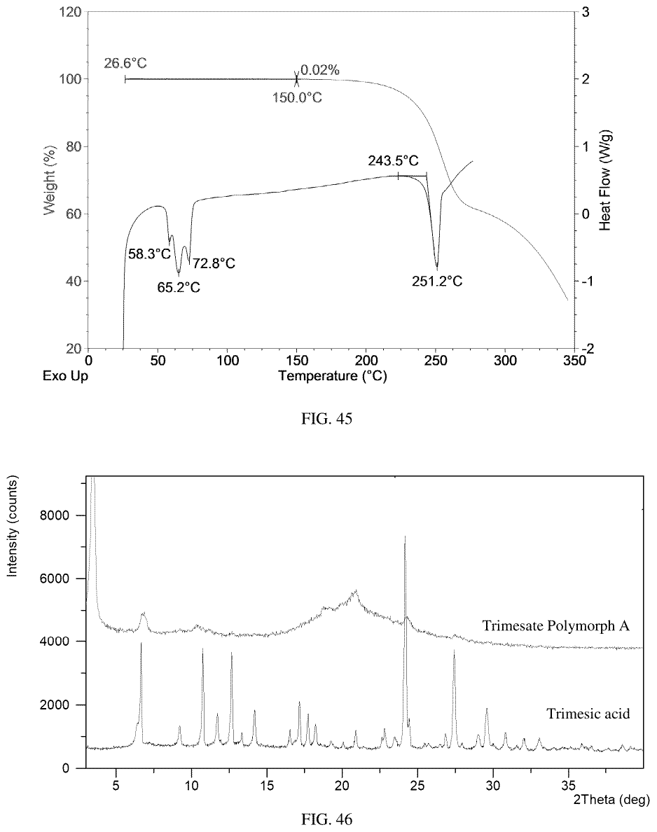

Compound 2 4-acetamido benzoate Polymorph A was prepared by combining Compound 2 freebase with 4-acetamido benzoic acid in n-heptane and showed crystallinity as characterized by XRPD in FIG. 44. The TGA/DSC data as shown in FIG. 45 indicate a weight loss of 0.02% up to 150° C. and multiple endothermic signals before decomposition.

### Example 3: Salts or Cocrystals of Compound 3

### Preparation

Compound 3 freebase, as characterized via modulated DSC (mDSC), exhibits no glass transition signal. A weight loss of 1.2% was observed up to 200° C., and endotherms were observed at −44.1° C. and −29.9° C. (peak).

Similar to the process described in Example 1 or 2, to identify a crystalline salt form or cocrystal of Compound 3, screening was performed under 93 conditions using 31 acids and three solvent systems. 0.5 mL stock solutions of Compound 3 freebase (~40 mg/mL) was dispersed in selected solvent and corresponding salt former was added with a molar charge ratio of 1:1. The mixtures of freebase and the coformer compound (e.g., an acid) were first transferred to temperature cycling from 50° C. to 5° C. for three cycles (heating rate of 4.5° C./min, cooling rate of 0.1 TC/min) and then stored at 5° C. before analysis. If the samples were still clear, they would be subjected to slow evaporation at 5° C. to obtain gels. Resulting solids were isolated and analyzed.

Isolated crystal solids were characterized by X-ray powder diffraction (XRPD), thermo-gravimetric analysis (TGA) and differential scanning calorimetry (DSC), with proton nuclear magnetic resonance (H NMR) to confirm the freebase chemical structure and also potential co-existence with some organic acids. Exemplary data from the initial findings are summarized in Table 7.

TABLE 7

| | | Solvent | | |
|---|---|---|---|---|
| # | Acid | n-Heptane | EtOAc | Toluene |
| 1 | Trimesic acid | Trimesate | Acid | Trimesate |
| | | Type A | | Type A |
| 2 | Trimellitic acid | Acid | Acid | Acid |
| 3 | (−)-2,3-Dibenzoyl-L- | Gel | Gel | Gel |
| | tartaric acid | | | |
| 4 | Fumaric acid | Gel | Gel | Gel |
| 5 | Terephthalic acid | Gel | Gel | Gel |
| 6 | Phthalic acid | Ge | Gel | Gel |
| 7 | Isophthalic acid | Acid | Acid | Acid |
| 8 | Benzoic acid | Gel | Gel | Gel |
| 9 | Cinnamic acid | Gel | Gel | Gel |
| 10 | 4-Hydroxy benzoic acid | Gel | Gel | Gel |
| 11 | Salicylic acid | Gel | Gel | Gel |
| 12 | Adipic acid | Acid | Acid | Acid |
| 13 | Suberic acid | Acid | Gel | Acid |
| 14 | Sebacic acid | Acid | Acid | Acid |
| 15 | 4-Acetamido benzoic | Acid | Acid | Acid |
| | acid | | | |
| 16 | S-(+)-Mandelic | Gel | Gel | Gel |
| 17 | Orotic acid | Acid | Acid | Acid |
| 18 | Hexanoic acid | Gel | Gel | Gel |
| 19 | Citric acid | Gel | Gel | Gel |
| 20 | Acetic acid | Gel | Gel | Gel |
| 21 | Succinic acid | Acid | Gel | Gel |
| 22 | Malonic acid | Gel | Gel | Gel |
| 23 | (+)-Camphor-10- | Gel | Gel | Gel |
| | sulfonic acid | | | |
| 24 | Nicotinic acid | Acid | Acid | Acid |
| 25 | (+)-L-tartaric acid | Gel | Gel | Gel |
| 26 | Hydrochloric acid | Gel | Gel | Gel |
| 27 | Sulfuric acid | Gel | Gel | Gel |
| 28 | Phosphoric acid | Gel | Gel | Gel |
| 29 | Methanesulfonic acid | Gel | Gel | Gel |
| 30 | p-Toluene sulfonic acid | Gel | Gel | Gel |
| 31 | 2,5-Dihydroxybenzoic | Gel | Gel | Gel |
| | acid | | | |

### Characterization of Trimesate

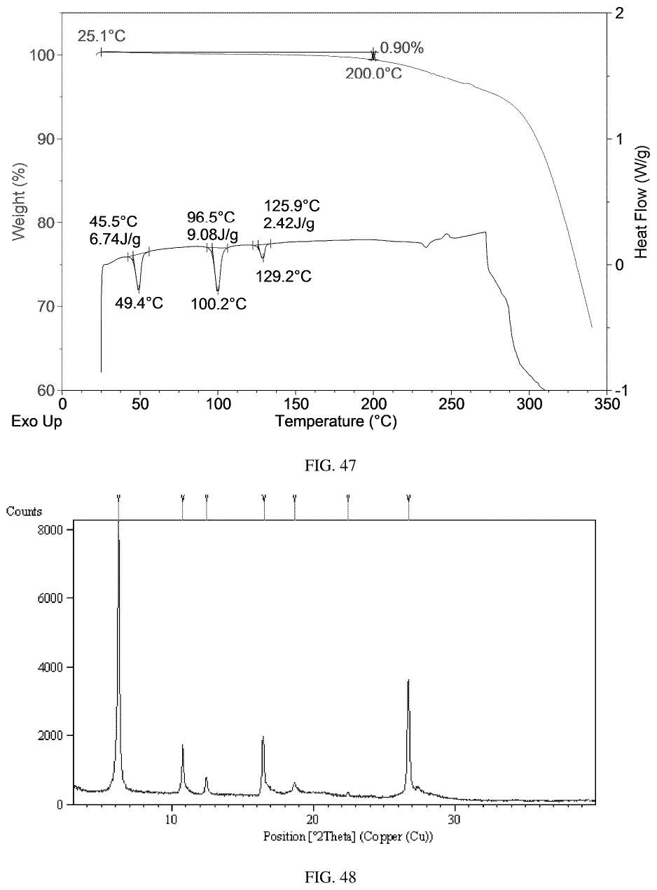

Compound 3 trimesate Polymorph A was prepared by combining Compound 3 freebase with trimesic acid in n-heptane and showed crystallinity as characterized by XRPD in FIG. 46. The TGA/DSC data as shown in FIG. 47 indicate a weight loss of 0.9% up to 200° C. and three endothermic peaks at 49.4° C., 100.2° C. and 129.2° C. (peak temperature) before decomposition. Polymorph B was obtained via temperature cycling in EtOH/n-heptane (1:19, v/v) from 50° C. to 5° C. with molar charge ratio (compound 3: trimesic acid) at 1:1, and showed crystallinity as characterized by XRPD in FIG. 48. The TGA/DSC data as shown in FIG. 49 indicate a weight loss of 5.4% up to 200° C. and two endothermic peaks at 239.9° C. and 257.5° C. before decomposition at 304.6° C. An ¹H NMR spectrum was collected using (CD₃)₂SO as the test solvent, and signals of trimesic acid and compound 3 were observed. See FIG. 50.

### Example 4: Salts or Co-Crystals of MC3

Only one crystalline salt of MC3 (O,O-Dibenzoyl-L-Tartrate, abbreviated as “DBLT” hereafter) has been previously identified, and only one polymorph, Type A, has been discovered for the DBLT salt. An onset temperature of 69.8° C. in DSC analysis indicated a low melting point, however, not as low as the free base which is oil-like at room temperature. The crude free base has an HPLC purity of 88.6 area % and was used in the synthesis of the DBLT salt. Impurities are not rejected by the salt formation and the purity of the crystallized salt was found to be the same as the crude free base. Additional salt screening experiments were performed to identify new crystalline salts.

An oil-like MC3 free base with an HPLC purity of 97.6 area % (“purified free base”) was used in the salt screening. A total of 24 acids and three solvent systems were screened. Crystalline salt hits were obtained with (+)-O,O-di-pivaloyl-D-tartaric acid (DPDT), (−)-O,O-di-pivaloyl-L-tartaric acid (DPLT), and trimesic acid.

### Solvent Screening

A solvent screening was performed by reaction of free base and DPDT, DPLT and trimesic acid in 17 selected solvents to improve crystallinity and facilitate salt isolation and re-preparation. The X-ray powder diffraction (XRPD) results showed that crystalline trimesate Type A and B were obtained in ketones, esters and some other selected solvents from slurry at room temperature. For DPDT and DPLT salts, no suitable anti-solvent was found, only clear solutions were obtained during the solvent screening.

Based on the screening results, attempts were made to re-prepare trimesate Type A and B, but only trimesate Type A was successfully prepared at a 100-mg scale. Both polymorphs were further characterized using thermogravimetric analysis (TGA), differential scanning calorimetry (DSC), polarizing microscopy (PLM), dynamic vapor sorption (DVS), and HPLC. The characterization results of trimesate samples are summarized in Table 8. As the results show, trimesate Type A is anhydrous and non-hygroscopic.

TABLE 8

| Salt form | Trimesate Type A | | Trimesate Type B |
|---|---|---|---|
| Prepared solvent | EtOAc | Cyclohexane | Toluene |
| Scale, mg | 100 | 100 | 10 |
| Molar ratio (acid/FB)^{a} | 1.2 | 1.1 | 1.5 |
| Speculated form^{b} | Anhydrate | Anhydrate | N/A |
| HPLC purity (area %) | 98.3 | 99.4^{c} | 93.7 |
| Weight loss (%) | 1.9 | 0.3 | 8.0 |
| Endotherm (° C., onset) | 186.4 | 183.8 | 186.8 |
| Hygroscopicity/purity decrease | Non-hygroscopic | N/A | N/A |
| Morphology | Aggregated of small particles (<20 μm) | | |
| Appearance of solution in preparation | Suspension | Wax/emulsus | Wax/emulsus |

N/A: not applicable or data not collected in this study.
^{a}the molar ratio (acid/FB) was determined by HPLC/IC.
^{b}results speculated based on the preliminary thermal analysis data.
^{c}average value of three sampling (100.0 area %, 99.34 area %, and 98.74 area %), suggesting the sample is inhomogeneous.
Hygroscopicity concluded using the water uptake up to 80% RH at 25° C.: <0.2% for non-hygroscopic.

### Salt Screening

A total of 41 screening experiments were designed based on the free base pKa >8 and the solubility of MC3. Crystalline hits of trimesate (Type A), DPDT and DPLT salts were obtained.

In the 1st tier experiments, about 10 mg of MC3 free base and the corresponding acid were mixed, at a 1:1 molar ratio, into a 1.5-mL glass vial and 0.5 mL of n-heptane were then added. The mixtures were stirred at room temperature for about two days. If clear solutions were obtained, the samples were cooled at 5° C. or left to evaporate to induce solid formation. All the obtained solids were isolated by centrifugation and vacuum dried at room temperature for about 5 hours before being analyzed by X-Ray Powder Diffraction (XRPD). As summarized in Table 9, amorphous salts or acids were found under most of the conditions while potential crystalline forms were obtained with DPDT, DPLT, and trimesic acid.

To enhance the chance of crystallization during the 2nd tier screening, the concentration of free base was increased from 20 to 50 mg/mL when using the acids that yielded solutions in the 1st tier screening. Also, isopropyl alcohol/n-heptane (3:97, v/v) was used with those acids which yielded crystalline acid in the 1st tier screening. As summarized in Table 10, no new crystalline hit was obtained.

Six more acids with structures closely related to trimesic acid were screened. The free base and the acids were mixed, at a 1:1 molar ratio, in EtOAc (free base loading 50 mg/mL) and the suspensions were then shaken at room temperature for about three days. The results are summarized in Table 11.

TABLE 9

| No. | Acid | Solid form |
|---|---|---|
| 1 | Hexanoic acid | Amorphousª |
| 2 | Fumaric acid | Acid |
| 3 | Adipic acid | Amorphous |
| 4 | Suberic acid | Acid |
| 5 | Sebacic acid | Acid |
| 6 | Alginic acid | Amorphousª |
| 7 | Cinnamic acid | Amorphousª |
| 8 | Benzoic acid, 4- | Acid |
| | acetamido | |
| 9 | (S)-(+)-Mandelic | Amorphousª |
| | acid | |
| 10 | (R)-(−)-Mandelic acid | Amorphousª |
| 11 | Benzyloxy lactic acid | Amorphousª |
| 12 | (+)-O,O-Di-pivaloyl-D- | DPDT salt Type |
| | tartaric acid | Aª |
| 13 | (−)-O,O-Di-pivaloyl-L- | DPLT salt Type |
| | tartaric acid | Aª |
| 14 | Terephthalic acid | Acid |
| 15 | Trimesic acid | Acid + new peaks^{c} |
| 16 | 4-Hydroxy benzoic | Acid |
| 17 | 2-(4-Hydroxybenzoyl)- | Amorphousª |
| | benzoic acid | |
| 18 | (+)-2,3-Dibenzoyl-D- | DBDT salt Type |
| | tartaric acid | A^{b} |

^{a}clear solution was observed after slurry at room temperature (RT) and 5° C., which was then transferred to slow evaporate at RT.
^{b}obtained in a previous experiment with no obvious purity improvement.
^{c}new peaks conformed to trimesate Type A.

TABLE 10

| No. | Acid | Solvent | Solid form |
|---|---|---|---|
| 1 | Hexanoic acid | n- | N/A |
| 2 | Alginic acid | Heptane | N/A |
| 3 | Cinnamic acid | | N/A |
| 4 | (S)-(+)-Mandelic acid | | N/A |
| 5 | R)-(−)-Mandelic acid | | N/A |
| 6 | Benzyloxy lactic acid | | N/A |
| 7 | (+)-O,O-Di-pivaloyl-D-tartaric acid | | N/A |
| 8 | (−)-O,O-Di-pivaloyl-L-tartaric acid | | N/A |
| 9 | 2-(4-Hydroxybenzoyl)-benzoic acid | | N/A |
| 10 | Fumaric acid | IPA/H₂O | Acid |
| 11 | Adipic acid | (3:97, | Amorphous |
| 12 | Suberic acid | v/v) | Acid |
| 13 | Sebacic acid | | Acid |
| 14 | Benzoic acid, 4-acetamido | | Acid |
| 15 | Terephthalic acid | | Acid |
| 16 | Trimesic acid | | Acid |
| 17 | 4-Hydroxy benzoic | | Acid |
| — | — | | — |

N/A: clear solution was observed after slurry at RT and 5° C.

TABLE 11

| No. | Acid | Solvent | Solid form |
|---|---|---|---|
| 1 | 1,2,4-Trimellitic acid | EtOAc | Amorphous |
| 2 | Phthalic acid | | Amorphous |
| 3 | Isophthalic acid | | Amorphous |
| 4 | Terephthalic acid | | Acid |
| 5 | Orotic acid | | Acid + new peaks* |
| 6 | 1,2,3-Benzene tricarboxylic acid | | Amorphous |

*only amorphous was observed in the re-preparation experiment.

### Optimization of Solvent Systems

A solvent screening was performed to select an optimal solvent system for re-preparation of the salt hits and to improve crystallinity. The free base was mixed in a 1:1 molar ratio, with DPDT, DPLT, and trimesic acid in 17 selected solvents. Trimesate Type A and B polymorphs were isolated from slurries in several solvents (see Table 12). DPDT and DPLT salts were not obtained as solids from any solvent. In addition, the samples containing tetrahydrofuran (THF)/H₂O, THF, cyclohexane and 1,4-dioxane were freeze-dried, but no crystalline solid was obtained.

TABLE 12

| Acid | | | | |
|---|---|---|---|---|
| Form | Solvent | DPDT | DPLT | Trimesic acid |
| 1 | Acetone | N/A* | N/A* | Trimesate Type A |
| 2 | Methyl isobutyl ketone (MIBK) | N/A | N/A | Trimesate Type A |
| 3 | Methyl ethyl ketone (MEK) | N/A | N/A | Trimesate Type A |
| 4 | CH₂Cl₂ | N/A | N/A | Acid |
| 5 | Methyl tert-butyl ether (MTBE) | N/A | N/A | Trimesate Type A |
| 6 | 2-Methyl tetrahydrofuran | N/A | N/A | N/A |
| | (2-MeTHF) | | | |
| 7 | Tetrahydrofuran (THF) | N/A* | N/A* | N/A |
| 8 | Anisole | N/A | N/A | Trimesate Type A |
| 9 | 1,4-Dioxane | N/A* | N/A* | N/A |
| 10 | EtOAc | N/A | N/A | Trimesate Type A |
| 11 | Isopropyl acetate (IPAc) | N/A | N/A | Trimesate Type A |
| 12 | Acetonitrile (CAN) | N/A* | N/A* | N/A |
| 13 | MeOH | N/A* | N/A* | N/A |
| 14 | Isopropyl alcohol (IPA) | N/A* | N/A* | N/A |
| 15 | Cyclohexane | N/A | N/A | Trimesate Type A |
| 16 | Xylene | N/A | N/A | N/A |
| 17 | Toluene | N/A | N/A | Trimesate Type B |

N/A: clear solution was obtained after slurry at RT and 5° C.
*about 0.2~0.3 mL of H₂O was added into the clear solution to induce precipitation and emulsion was obtained.

### Preparation of Trimesate Polymorphs (100 mg Scale)

Heating and cooling experiments were carried out at 100-mg scale to improve crystal morphology and chemical purity. Trimesate Type A polymorph was successfully re-prepared in cyclohexane and EtOAc following the procedure detailed below.

### Preparation of Trimesate Type A Polymorph:

A 5 mL vial was charged with 100.0 mg of the free base (97.6 area %) and 30 mg of trimesic acid and 2 mL of cyclohexane or EtOAc, were added. The suspension was stirred at room temperature for about 0.5 h. The solution was continued to be stirred while being heated and cooled between 5° C. and 50° C. for two cycles with a 4.5° C./min heating rate and a 0.1° C./min cooling rate. The resulting solid was isolated by centrifugation and dried under vacuum at room temperature for 2 hours before characterization.

### Preparation of Trimesate Type B Polymorph:

About 10 mg of free base and trimesic acid were mixed, at a 1:1 molar ratio, in a 1.5-mL glass vial. n-Heptane (0.5 mL) was added. The mixtures were magnetically stirred at RT for about two days. If clear solutions were obtained, the samples were cooled at 5° C. or left to evaporate to induce solid formation. All the obtained solids were isolated by centrifugation and vacuum dried at RT for about 5 hours before being analyzed by XRPD.

### Characterization of Trimesate Polymorphs

Both trimesate Type A (100-mg scale) and Type B (10-mg scale) were characterized, and results are summarized in Table 8.

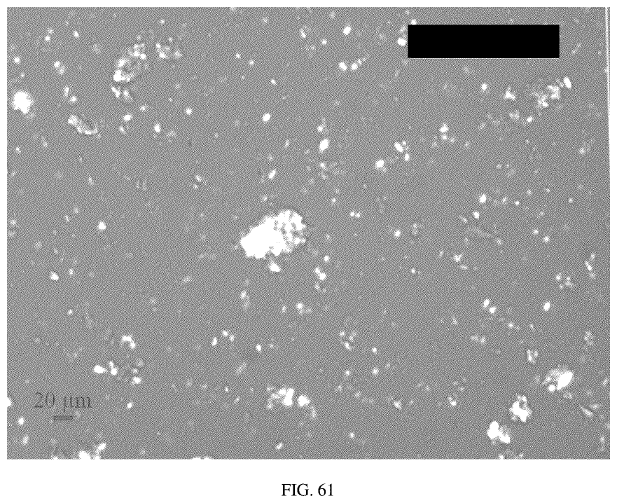

The XRPD pattern of polymorph A is shown in FIG. 52. TGA/DSC curves of trimesate Type A polymorph prepared with cyclohexane, displayed in FIG. 53, shows a weight loss of 0.3% before 120° C. and a sharp melting endotherm at 183.8° C. (onset temperature). The TGA/DSC curves of trimesate Type A polymorph prepared with EtOAc displayed in FIG. 54, shows a weight loss of 1.9% before 120° C. and a sharp melting endotherm at 186.4° C. (onset temperature). Agglomerate and small particles (<20 μm) were observed in the trimesate Type A polymorphs. See FIGS. 55 and 56. The XRPD pattern of trimesate Type B polymorph is shown in FIG. 59. TGA/DSC curves displayed in FIG. 60 show a weight loss of 8.0% before 150° C. and a sharp melting endotherm at 186.8° C. (onset temperature). As shown in FIG. 61, agglomerate particles with small size (<20 μm) are observed in trimesate Type B sample.

As the DVS result shows, the trimesate Type A polymorph is non-hygroscopic. See FIG. 57. The hygroscopicity of free base (crude and pure) was determined as well. The crude free base was slightly hygroscopic (0.27 and 0.24% water uptake at 80% relative humidity for the desorption and adsorption isotherms, respectively), but the pure free base was non-hygroscopic (0.17 and 0.14% water uptake at 80% relative humidity for the desorption and adsorption isotherms, respectively).

### HPLC Purity of Trimesate Type A

Trimesate Type A samples were prepared according to the procedure described in the foregoing, using the crude free base (HPLC purity of 88.5 area %) or purified free base (HPLC purity of 97.6 area %) as starting material, and analyzed by HPLC. The results of the HPLC purity analysis for the samples prepared with crude and purified free base are summarized in Tables 13 and 14, respectively. No significant HPLC purity change was observed for both samples after the DVS experiment.

TABLE 13

| | | Imp 1 | Imp 2 | Imp 3 | Imp 4 | Imp 5 | Imp 6 |
|---|---|---|---|---|---|---|---|
| | Solvent/scale | (RRT | (RRT | (RRT | (RRT | (RRT | (RRT |
| Sample | (mg) | 0.08) | 0.50) | 0.51) | 0.52) | 0.53) | 0.90) |
| Free base | N/A | 0.11 | 0.22 | <0.05 | 0.34 | 0.44 | 1.74 |
| Trimesate | EtOAc/100 | <0.05 | 4.18 | 1.38 | <0.05 | <0.05 | 1.96 |
| Type A | Cyclohexane/100 | <0.05 | <0.05 | <0.05 | <0.05 | <0.05 | 1.91 |
| | | Imp 7 | Imp 8 | Imp 9 | Imp 10 | Imp 11 | |
| | Solvent/scale | (RRT | (RRT | RRT | (RRT | (RRT | Area |
| Sample | (mg) | 0.91) | 0.99) | 1.04) | 1.06) | 1.14) | (%) |
| Free base | N/A | 0.16 | 0.36 | 5.02 | 0.28 | 2.74 | 88.6 |
| Trimesate | EtOAc/100 | <0.05 | <0.05 | 3.78 | <0.05 | 3.31 | 85.38 |
| Type A | Cyclohexane/100 | <0.05 | <0.05 | 4.45 | <0.05 | 3.97 | 89.66 |

TABLE 14

| | | Imp 1 | Imp 2 | Imp 3 | | |
|---|---|---|---|---|---|---|
| | Solvent/scale | (RRT | (RRT | (RRT | | |
| Sample | (mg) | 0.58) | 1.04) | 1.14) | Area(%) | |
| Free base | N/A | 0.99 | 1.41 | <0.05 | 97.60 | |
| Trimesate | EtOAc/100 | <0.05 | 1.04 | 0.68 | 98.28 | |
| Type A | Cyclohexane/ | <0.05 | <0.05 | <0.05 | 100.00 | 99.36 |
| | 100 | <0.05 | 1.26 | <0.05 | 98.74 | (av.) |
| | | <0.05 | 0.66 | <0.05 | 99.34 | |

The entire disclosure of each of the patent documents and scientific articles referred to herein is incorporated by reference for all purposes.

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1-38. (canceled)

39. A salt or cocrystal of heptadecan-9-yl 8-((2-hydroxyethyl) (8-(nonyloxy)-8-oxooctyl)amino) octanoate (“Compound 3”) and trimesic acid, wherein the salt or cocrystal exhibits an X-ray powder diffraction pattern obtained using Cu Kα radiation, comprising two, three, four, or more characteristic peaks expressed in degrees 2-theta (+/−0.2) selected from the group consisting of 6.2, 10.8, 12.4, 16.5, 18.7, 22.5, and 26.7.

40. The salt or cocrystal of claim 39, wherein the salt or cocrystal exhibits an X-ray powder diffraction pattern obtained using Cu Kα radiation, having characteristic peaks expressed in degrees 2-theta (+/−0.2) at 6.2, 10.8, 16.5, and 26.7.

41. The salt or cocrystal of claim 39, wherein the salt or cocrystal exhibits an X-ray powder diffraction pattern obtained using Cu Kα radiation, having at least five characteristic peaks expressed in degrees 2-theta (+/−0.2), selected from the group consisting of 6.2, 10.8, 12.4, 16.5, 18.7, 22.5 and 26.7.

42. The salt or cocrystal of claim 39, wherein the salt or cocrystal exhibits an X-ray powder diffraction pattern obtained using Cu Kα radiation, having at least six characteristic peaks expressed in degrees 2-theta (+/−0.2), selected from the group consisting of 6.2, 10.8, 12.4, 16.5, 18.7, 22.5 and 26.7

43. (canceled)

44. The salt or cocrystal of claim 39, wherein the salt or cocrystal exhibits an X-ray powder diffraction pattern obtained using Cu Kα radiation, having characteristic peaks expressed in degrees 2-theta (+/−0.2) at 6.2, 10.8, 12.4, 16.5, 18.7, 22.5, and 26.7.

45. The salt or cocrystal of claim 39, wherein the salt or cocrystal exhibits a differential scanning calorimetry thermogram showing a primary endotherm expressed in units of ° C. at a temperature of 305+/−2° C.

46-47. (canceled)

48. The salt or cocrystal of claim 39, wherein said salt or cocrystal is substantially free of impurities.

49. The salt or cocrystal of claim 39, wherein said salt or cocrystal is a crystalline solid substantially free of other crystalline forms of the salt or cocrystal.

50-94. (canceled)

95. The salt or cocrystal of claim 39, wherein the stoichiometry of Compound 3 and trimesic acid is within the range of from 1:0.2 to 1:5 or from 1:0.5 to 1:2.

96. The salt or cocrystal of claim 39, wherein the stoichiometry of Compound 3 and trimesic acid is about 1:1.

97. The salt or cocrystal of claim 39, being an anhydrate, a solvate, or a hydrate.
